SEQUENCE LISTING (42 sequences)

SEQ ID NO: 1 - DNA, 20 bp, synthetic construct
  source 1..20: mol_type = other DNA
ggaatggataccgaacaacg

SEQ ID NO: 2 - DNA, 20 bp, synthetic construct
  source 1..20: mol_type = other DNA
caacgctagtctcagcagca

SEQ ID NO: 3 - DNA, 20 bp, synthetic construct
  source 1..20: mol_type = other DNA
gcgactgcagactcgtacac

SEQ ID NO: 4 - DNA, 20 bp, synthetic construct
  source 1..20: mol_type = other DNA
tctcgttgacccaaaggttc

SEQ ID NO: 5 - DNA, 19 bp, synthetic construct
  source 1..19: mol_type = other DNA
gccatgttcaggaggaagg

SEQ ID NO: 6 - DNA, 19 bp, synthetic construct
  source 1..19: mol_type = other DNA
ctcggtgaactccatctcg

SEQ ID NO: 7 - DNA, 20 bp, synthetic construct
  source 1..20: mol_type = other DNA
gagaatgcagacgcccaagc

SEQ ID NO: 8 - DNA, 22 bp, synthetic construct
  source 1..22: mol_type = other DNA
ctggagcttgcagtcgttgatc

SEQ ID NO: 9 - DNA, 20 bp, synthetic construct
  source 1..20: mol_type = other DNA
acctgtcagttgggatgaaa

SEQ ID NO: 10 - DNA, 20 bp, synthetic construct
  source 1..20: mol_type = other DNA
gcacgagtttgaggcataat

SEQ ID NO: 11 - DNA, 24 bp, synthetic construct
  source 1..24: mol_type = other DNA
ccgttctgtccctctacgctagtg

SEQ ID NO: 12 - DNA, 24 bp, synthetic construct
  source 1..24: mol_type = other DNA
ggaactgctctttgcactctcgag

SEQ ID NO: 13 - DNA, 20 bp, synthetic construct
  source 1..20: mol_type = other DNA
tacaatggaggcatccaaca

SEQ ID NO: 14 - DNA, 20 bp, synthetic construct
  source 1..20: mol_type = other DNA
ctgttttggggagtgaggta

SEQ ID NO: 15 - DNA, 20 bp, synthetic construct
  source 1..20: mol_type = other DNA
ggcttggcttaaaggaaggt

SEQ ID NO: 16 - DNA, 21 bp, synthetic construct
  source 1..21: mol_type = other DNA
tcagtccaacttccagaatgg

SEQ ID NO: 17 - DNA, 20 bp, synthetic construct
  source 1..20: mol_type = other DNA
tgacatgggacaacaggcta

SEQ ID NO: 18 - DNA, 20 bp, synthetic construct
  source 1..20: mol_type = other DNA
cagctaggttttcgccgtag

SEQ ID NO: 19 - DNA, 20 bp, synthetic construct
  source 1..20: mol_type = other DNA
gatggtcagacccgtgaaca

SEQ ID NO: 20 - DNA, 20 bp, synthetic construct
  source 1..20: mol_type = other DNA
ccttggagtacttcggggtg

SEQ ID NO: 21 - DNA, 20 bp, synthetic construct
  source 1..20: mol_type = other DNA
gtgtccgagaggccagacta

SEQ ID NO: 22 - DNA, 20 bp, synthetic construct
  source 1..20: mol_type = other DNA
cattgttgcaacgagcccga

SEQ ID NO: 23 - DNA, 22 bp, synthetic construct
  source 1..22: mol_type = other DNA
ttggttttgcaccactgacttc

SEQ ID NO: 24 - DNA, 21 bp, synthetic construct
  source 1..21: mol_type = other DNA
aagctctggcactgtcaaagc

SEQ ID NO: 25 - DNA, 26 bp, synthetic construct
  source 1..26: mol_type = other DNA
tcccagaaacattatacattgctgat

SEQ ID NO: 26 - DNA, 20 bp, synthetic construct
  source 1..20: mol_type = other DNA
gtgtccgagaggccagacta

SEQ ID NO: 27 - DNA, 20 bp, synthetic construct
  source 1..20: mol_type = other DNA
ggacttgttgcactcgttgt

SEQ ID NO: 28 - DNA, 22 bp, synthetic construct
  source 1..22: mol_type = other DNA
catgagatctctgtgctttgcg

SEQ ID NO: 29 - DNA, 20 bp, synthetic construct
  source 1..20: mol_type = other DNA
tatccccaccgagtctgttc

SEQ ID NO: 30 - DNA, 22 bp, synthetic construct
  source 1..22: mol_type = other DNA
tgatggcacgtctgaattaccc

SEQ ID NO: 31 - DNA, 20 bp, synthetic construct
  source 1..20: mol_type = other DNA
ttctctcgctcaagactcgc

SEQ ID NO: 32 - DNA, 20 bp, synthetic construct
  source 1..20: mol_type = other DNA
atcaggaggctccacaaagc

SEQ ID NO: 33 - DNA, 20 bp, synthetic construct
  source 1..20: mol_type = other DNA
gcaggtgactaattcgggct

SEQ ID NO: 34 - DNA, 30 bp, synthetic construct
  source 1..30: mol_type = other DNA
aatgaccttaacaagttctgataccactaa

SEQ ID NO: 35 - DNA, 20 bp, synthetic construct
  source 1..20: mol_type = other DNA
cattgttgcaacgagcccga

SEQ ID NO: 36 - DNA, 20 bp, synthetic construct
  source 1..20: mol_type = other DNA
gacacttgtgcttgccatga

SEQ ID NO: 37 - DNA, 23 bp, synthetic construct
  source 1..23: mol_type = other DNA
atcctttccatgcaagagtcagt

SEQ ID NO: 38 - DNA, 20 bp, synthetic construct
  source 1..20: mol_type = other DNA
tctggttttcccattccttg

SEQ ID NO: 39 - DNA, 24 bp, synthetic construct
  source 1..24: mol_type = other DNA
aaccgttttccaagttccacagtt

SEQ ID NO: 40 - DNA, 20 bp, synthetic construct
  source 1..20: mol_type = other DNA
tgatgccactcaagtcaggg

SEQ ID NO: 41 - DNA, 20 bp, synthetic construct
  source 1..20: mol_type = other DNA
aatgcagaggctcccttgtc

SEQ ID NO: 42 - DNA, 20 bp, synthetic construct
  source 1..20: mol_type = other DNA
tgtgcatgtgctagaagcgt

(19) United States
(12) Patent Application Publication
(10) Pub. No.: US 2026/0182579 A1
(43) Pub. Date: Jul. 2, 2026

(54) ENZYMATICALLY PRODUCED BIOPESTICIDES

(71) Applicant: ZYMtronix Catalytic Systems, Inc., Cornell University, NY (US)

(72) Inventors: Alexander Chris Hoepker, Ithaca, NY (US); Hilary Mayton, Ithaca, NY (US); Dezi Elzinga, Ithaca, NY (US)

(21) Appl. No.: 19/130,664

(22) PCT Filed: Nov. 17, 2023
(86) PCT No.: PCT/US2023/080354

§ 371 Date: May 16, 2025

Related U.S. Application Data
(60) Provisional application No. 63/426,661, filed on Nov. 18, 2022.

(51) Int. Cl. A01N 59/12; A01N 25/04; A01N 59/00; A01N 63/50; A01P 21/00

(52) U.S. Cl. CPC A01N 59/12; A01N 25/04; A01N 59/00; A01N 63/50; A01P 21/00; C12Y 101/03004; C12Y 111/01007

(57) ABSTRACT

The present invention provides compositions and methods for reducing microbial, nematodal, and oomycotic contamination or infection on all parts of plants and products therefrom. In particular, the invention provides antimicrobial or biostatic enzymes (either singularly or in combinations) in formulations optimized for eliciting a Systemic Acquired Resistance (SAR) in plants. The invention also provides compositions that elicit activation of genes associated with flowering and fruit development. The invention further provides compositions with biostimulating effects on plants. In some embodiments, compositions are dormant and become active upon exposure to hydration, oxygen, or mixing.

### CROSS REFERENCE TO RELATED APPLICATIONS

This paragraph is intentionally left blank.

### FIELD OF THE INVENTION

The present invention provides compositions and methods for reducing microbial, nematodal, and oomycotic contamination or infection on all parts of plants and products therefrom. In particular, the invention provides antimicrobial or biostatic enzymes (either singularly or in combinations) in formulations optimized for eliciting a Systemic Acquired Resistance (SAR) in plants. The invention also provides compositions that elicit activation of genes associated with flowering and fruit development. The invention further provides compositions with biostimulating effects on plants. In some embodiments, compositions are dormant and become active upon exposure to hydration, oxygen, or mixing.

### BACKGROUND OF THE INVENTION

To feed a growing population of 10 billion in 2050, agricultural production will need to increase by 70% over the next 30 years. Critical to this mission will be the development of innovative plant protection tools and strategies that help mitigate pests and diseases to preserve the world food supply. Plant diseases are estimated to cost the global economy $220B, and losses due to invasive insects total $70B. In total, 20-40% of crops are lost to pathogens worldwide. Between 2009 and 2010, fungal infections globally caused losses in five major crops (rice, wheat, maize, potatoes, and soybean) that could have fed 8.5% of the world population.

Contaminating and infectious microorganisms significantly reduce the yield, quality, and safety of agricultural products worldwide. Current methods for reducing plant and animal infections rely heavily on the use of antimicrobial chemicals that may result in fungicide and antibiotic resistance that, in turn, increases the probability of selecting for drug-resistant plant, animal, and human pathogens. These microbes have been selected to survive in the presence of medically and agriculturally important antimicrobial chemicals and are a significant threat to human health and food security.

For example, Antibiotic Resistant Microbes (ARM) are a growing public health concern because infections have become increasingly difficult and expensive to treat. Concern turns into crisis in hospital environments. Antibiotics of last resort such as vancomycin, are steadily becoming ineffectual against superstrains. Carbapenem Resistant

Enterobacteriaceae (CRE), some of the most ubiquitous microbes in the environment, are now resistant to almost all antibiotics. CRE infections are so difficult to treat that 50% of patients infected by them die. In the 2013 Antibiotic Resistance Threat report, the CDC identifies three major concerns: 1) new active molecules are harder to discover and produce, 2) development costs are prohibitive, and 3) resistance spreads faster than ever. The World Health Organization (WHO) warns that “[i]n the absence of urgent corrective and protective actions, the world is heading toward a post-antibiotic era, in which many common infections will no longer have a cure and [will], once again, kill unabated.” (World Health Day, Combat Drug Resistance: No Action Today Means No Cure Tomorrow, Statement by WHO Director-General, Dr. Margaret Chan, Apr. 6, 2011, http://www.who.int/mediacentre/news/statements/2011/whd_20110407/en/.)

In 2013, the Center for Disease Control (CDC) estimated that 70 percent of the bacteria that caused hospital-acquired infections were resistant to at least one of the relevant antibiotics. (Antibiotic Resistance Threats in the United States, 2013, Centers for Disease Control and Prevention: Atlanta, GA, http://www.cdc.gov/drugresistance/threat-report-2013/.) It has long been argued by public advocacy groups, such as the Alliance for the Prudent Use of Antibiotics, that antibiotics are societal drugs. Individual use affects the entire community.

Such antibiotic resistance has become a worldwide concern now that the consequences of antibiotic overuse are being studied and reported. The CDC now estimates that in the U.S. at least 23,000 people die from multiple antibiotic-resistant bacteria infections every year. Id. In the U.S. alone, these superbug infections are responsible for $20 billion in excess healthcare costs, $35 billion in societal costs, and 8 million additional hospital stays each year. (Roberts et al., Clin. Infect. Dis. 49 (8): 1175-84 (2009).)

Nematodes are microscopic worms that cause eighty billion dollars of crop loss in the world each year. Plant-parasitic nematodes threaten crops throughout the world. In fact, all crops are damaged by at least one species of nematode. They attack almost every part of the plant including roots, stems, leaves, fruits and seeds.

Oomycetes, or water molds, are fungal-like eukaryotes classified as stramenopiles. They are phylogenetically grouped with diatoms and brown algae. They are among the most problematic group of disease-causing organisms in both agriculture and aquaculture. They represent a recurrent threat for global food security. Oomycetes cause some of the most devastating plant diseases affecting crops, ornamental plants and trees. They result in major economic losses and serious damage to natural ecosystems. The most notorious species are members of the genus Phytophthora. For instance, the late blight pathogen Phytophthora infestans triggered the Irish potato famine. Other notable species include Phytophthora palmivora which causes cocoa black pod and the sudden oak death pathogen Phytophthora ramorum that threatens native tree species. Additional important oomycete plant pathogens include members of the Pythium genus and downy mildews.

Other nonlimiting examples of devastating pathogens include Pseudoperonospora cubensis, causal agent of downy mildew, Podosphaera xanthii, causal agent of powdery mildew, and Xanthamonas campestris, causative agent of black rot.

Controlling plant pathogens relies heavily on synthetic chemicals to maintain high product yields. The public has shown increasing concern, however, over the effects that agrochemical residues have on human health and the environment. (Mark et al., FEMS Microbiol. Ecol. 56 (2): 167-77 (2006); Ritter et al., J. Tox. Environ. Health 9 (6): 441-56 (2006).) Farmers who use synthetic agrochemicals have more neurological problems that include headaches, fatigue, insomnia, dizziness and hand tremors. (http://www.niehs.nih.gov/health/topics/agents/pesticides/). Agrochemicals may also cause birth defects, nerve damage, cancer, decreased sperm motility and acute poisoning (Moses, AAOHN J., 37 (3): 115-30 (1989); Reeves and Schafer Int'l J., Occup. Environ. Health 9 (1): 30-39 (2003); Carozza et al., Environ. Health Perspect. 116 (4): 559-65 (2008); U.S. Environmental Protection Agency, 2014, http://www.epa.gov/pesticides/food/risks.htm). Furthermore, protecting crops from fungal pathogens is particularly challenging for organic crops on which synthetic antifungal chemicals cannot be used.

Fungicides and antibiotics are widely used in developed agricultural systems to control disease and safeguard crop yield and quality. Over time, however, resistance to many of the most effective fungicides and antibiotics has emerged and spread in pathogen populations (Lucas et al., Adv Appl Microbiol., 90:29-92 (2015)). The widespread practice of routinely dosing farm animals with antifungals and antibiotics is contributing to this threat. Much of this use is for preventing, rather than treating, disease. Drug-resistant microbes carried by farm animals can spread to humans through consumption of contaminated food, from direct contact with animals, or by environmental spread, for example, in contaminated water or soil. Antibiotic and fungicide resistant pathogens of humans and farm animals are emerging and spreading at a rate that may not be contained by the development of new drugs.

Thus there is a significant need for new methods of controlling fungal, bacterial, oomycete, and nematode pathogens that cause agricultural contamination.

### SUMMARY OF THE INVENTION

The present invention provides compositions and methods for reducing microbial, nematodal, and oomycotic contamination or infection on all parts of plants and products therefrom. In particular, the invention provides antimicrobial or biostatic enzymes (either singularly or in combinations) in formulations optimized for eliciting a Systemic Acquired Resistance (SAR) in plants. The invention also provides compositions that elicit activation of genes associated with flowering and fruit development. The invention further provides compositions with biostimulating effects on plants. In some embodiments, compositions are dormant and become active upon exposure to hydration, oxygen, or mixing.

Thus, the invention provides a composition, comprising a hydrogen peroxide source and an iodide source, wherein the composition is optimized for inducing a Systemic Acquired Resistance (SAR) in a plant. In some embodiments, the composition minimizes phytotoxicity. In some embodiments, the hydrogen peroxide source is dissolved hydrogen peroxide. In other embodiments, the hydrogen peroxide source is glucose oxidase (GOx) and glucose. In some embodiments, the GOx is at a concentration of about 1, 2, 3, 4, 5, 6, 7, 8, 9, 10, 11, 12, 13, 14, 15, 16, 17, 18, 19, 20, 30, 40, or 50 U/ml. In some embodiments, the GOx is at a concentration that ranges from about 1 to 50 U/ml. In preferred embodiments, the GOx is at a concentration of about 10 U/ml, 20 U/ml, or 40 U/ml. In other embodiments, the glucose is at a concentration of about 2, 4, 6, 8, 10, 20, 30, 40, 50, 60, 70, 80, 90, 100, 200, 300, 400, or 500 mM. In some embodiments, the glucose is at a concentration that ranges from about 10 to 500 mM. In other embodiments, the glucose is at a concentration that ranges from about 10 to 100 mM. In preferred embodiments, the glucose is at a concentration of 50 mM.

In some embodiments of the invention, the iodide source is an iodide salt. In other embodiments, the iodide salt is KI, NaI, or NH₄I. In other embodiments, the iodide salt is KI. In other embodiments, the iodide salt has a concentration of about 0.05, 0.1, 0.2, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 20.0, 30.0, 35.0, 40.0, 50.0, 60.0, 70.0, 80.0, 90.0, or 100.0 mM. In some embodiments, the iodide salt is at a concentration that ranges from about 0.05 to 100 mM. In other embodiments, the iodide salt is at a concentration that ranges from about 0.05 to 5 mM. In other embodiments, the iodide salt is at a concentration that ranges from about 10 to 80 mM. In preferred embodiments, the iodide salt has a concentration of about 2 mM. In other preferred embodiments, the KI is at a concentration of about 2 mM. In other preferred embodiments, the iodide salt has a concentration of about 2 mM, 4 mM, 18 mM, 35 mM, 40 mM, or 70 mM.

In some embodiments of the invention, composition the compositions as disclosed herein do not comprise lactoperoxidase. In other embodiments, the composition does not comprise a thiocyanate source.

In some embodiments of the invention, compositions as disclosed herein further comprising a thiocyanate source. In other embodiments, the thiocyanate source is a thiocyanate salt or an organic thiocyanate. In other embodiments, the thiocyanate salt is KSCN, NaSCN, or NH₄SCN. In other embodiments, the thiocyanate salt is derived from glucosinolates, from chemical sources, or from enzymatic reactions.

In some embodiments, the thiocyanate salt has a concentration of about 0.5, 0.6, 0.7, 0.8, 0.9, 1.0, 1.5, 2.0, 2.5, 3.0, 3.5, 4.0, 5.0, 6.0, 7.0, 8.0, 9.0, 10.0, 11.0, 12.0, 13.0, 14.0, 15.0, 16.0, 17.0, 18.0, 19.0, or 20.0 mM. In other embodiments, the thiocyanate salt is at a concentration that ranges from about 0.5 to 20.0 mM. In other embodiments, the thiocyanate salt is at a concentration that ranges from about 0.5 to 5.0 mM. In other embodiments, the thiocyanate salt is at a concentration that ranges from about 0.5 to 1.0 mM.

In some embodiments, the ratio of iodide salt to thiocyanate is about 10:1, 5:1, 2:1, 1:1, 0.5:1, or 0.2:1.

In some embodiments of the invention, the hydrogen peroxide source is GOx at a concentration of 20 U/ml and glucose at a concentration of 300 mM, and the KI is at a concentration of 70 mM. In other embodiments, the hydrogen peroxide source is GOx at a concentration of 40 U/ml and glucose at a concentration of 300 mM, and the KI is at a concentration of 70 mM. In other embodiments, the hydrogen peroxide source is GOx at a concentration of 20 U/ml and glucose at a concentration of 300 mM, and the KI is at a concentration of 18 mM. In other embodiments, the hydrogen peroxide source is GOx at a concentration of 20 U/ml and glucose at a concentration of 300 mM, and the KI is at a concentration of 35 mM. In other embodiments, the hydrogen peroxide source is GOx at a concentration of 40 U/ml and glucose at a concentration of 300 mM, and the KI is at a concentration of 35 mM. In other embodiments, the hydrogen peroxide source is GOx at a concentration of 10 U/ml and glucose at a concentration of 50 mM, and the KI is at a concentration of 2 mM. In other embodiments, the hydrogen peroxide source is GOx at a concentration of 10 U/ml and glucose at a concentration of 50 mM, and the KI is at a concentration of 4 mM.

In some embodiments of the invention, the compositions as disclosed herein act as a biostimulant. In other embodiments, the composition as disclosed herein directly kill microbes.

In some embodiments of the invention, the compositions as disclosed herein further comprise lactoperoxidase (LP). In some embodiments the LP is at a concentration of about 1, 3, 5, 10, 11, 12, 13, 14, 15, 16, 17, 18, 19, 20, 21, 22, 23, 24, 25, 26, 27, 28, 29, 30 35, 40, 45, or 50 U/ml.

In some embodiments of the invention, the composition is dormant and becomes active upon exposure to hydration, oxygen, or mixing.

The invention provides a method of inducing Systemic Acquired Resistance (SAR) in a plant, comprising the step of administering a biocidal composition as disclosed herein at a rate that is optimized for SAR induction. In some embodiments, the method minimizes phytotoxicity. In other aspects, the biocidal composition is administered by coating, spraying, sprinkling, atomizing, overhead spraying, watering, immersing, overhead irrigation, or drip irrigation.

In other aspects of the invention, the plant is selected from the group consisting of vegetable, fruit, flower, and field crop.

In some aspects, the vegetable plant is selected from the group consisting of tomato, pea, onion, garlic, parsley, oregano, basil, cilantro, carrot, cabbage, cucumber, radish, pepper, broccoli, cauliflower, spinach, kale, chard, artichoke, and lettuce.

In other aspects, the fruit plant is selected from the group consisting of citrus, tomato, orange, lemon, lime, avocado, clementine, apple, persimmon, pear, peach, nectarine, berry, strawberry, raspberry, grape, blueberry, blackberry, cherry, apricot, gourds, squash, zucchini, eggplant, pumpkin, coconut, guava, mango, papaya, melon, honeydew, cantaloupe, watermelon, banana, plantain, pineapple, quince, sorbus, loquata, plum, currant, pomegranate, fig, olive, fruit pit, a nut, peanut, almond, cashew, hazelnut, brazil nut, pistachio, and macadamia.

In other aspects, the flower plant is selected from the group consisting of annual, perennial, bulb, flowering woody stem, carnation, rose, tulip, poppy, snapdragon, lily, mum, iris, alstroemeria, pom, fuji, and bird of paradise.

In other aspects, the field crop is selected from the group consisting of corn, wheat, soybean, canola, sorghum, potato, sweet potato, yam, lentils, beans, snap beans, cassava, coffee, hay, buckwheat, oat, barley, rape, switchgrass, elephant grass, beet, sugarcane, and rice.

In other aspects, the biocidal compositions are administered to plant leaves or seeds.

The invention provides a method for inducing iodine fortification in a plant, comprising the step of administering a biocidal composition as disclosed herein at a rate that is optimized for increasing iodine uptake or retention and induction and, in some embodiments, minimizing phytotoxicity. In some aspects, the biocidal composition is administered by coating, spraying, sprinkling, atomizing, overhead spraying, watering, immersing, overhead irrigation, or drip irrigation. In other aspects, the plant is selected from the group consisting of vegetable, fruit, flower, and field crop.

In some aspects, the vegetable plant is selected from the group consisting of tomato, pea, onion, garlic, parsley, oregano, basil, cilantro, carrot, cabbage, cucumber, radish, pepper, broccoli, cauliflower, spinach, kale, chard, artichoke, and lettuce.

In other aspects, the fruit plant is selected from the group consisting of citrus, tomato, orange, lemon, lime, avocado, clementine, apple, persimmon, pear, peach, nectarine, berry, strawberry, raspberry, grape, blueberry, blackberry, cherry, apricot, gourds, squash, zucchini, eggplant, pumpkin, coconut, guava, mango, papaya, melon, honeydew, cantaloupe, watermelon, banana, plantain, pineapple, quince, sorbus, loquata, plum, currant, pomegranate, fig, olive, fruit pit, a nut, peanut, almond, cashew, hazelnut, brazil nut, pistachio, and macadamia.

In other aspects, the flower plant is selected from the group consisting of annual, perennial, bulb, flowering woody stem, carnation, rose, tulip, poppy, snapdragon, lily, mum, iris, alstroemeria, pom, fuji, and bird of paradise.

In other aspects, the field crop is selected from the group consisting of corn, wheat, soybean, canola, sorghum, potato, sweet potato, yam, lentils, beans, snap beans, cassava, coffee, hay, buckwheat, oat, barley, rape, switchgrass, elephant grass, beet, sugarcane, and rice.

In other aspects of the invention, the biocidal compositions are administered to plant leaves or seeds.

The invention provides a method of manufacturing an enzyme-free biocidal composition, comprising:
a) immobilizing an H₂O₂-producing enzyme and a free radical producing (FRP) enzyme in a reaction container,
b) exposing the immobilized H₂O₂-producing and free radical producing (FRP) enzymes to a reaction solution comprising an iodide source, a thiocyanate source, and a substrate for the H₂O₂-producing enzyme; and
c) collecting a product solution, wherein the solution comprises reactive oxidative species.

In some aspects, the H₂O₂-producing enzyme is glucose oxidase (GOx) and the substrate is glucose. In other aspects, the FRP enzyme is lactoperoxidase. In other aspects, the iodide source is KI. In other aspects, the thiocyanate source is KSCN. In preferred aspects, the glucose is at a concentration of 300 mM in the solution. In other preferred aspects, the KI is at a concentration of 35 mM in the solution. In other preferred aspects, the KSCN is at a concentration of 8 mM in the solution.

In some aspects of the invention, the product solution comprises one or more reactive oxidative species selected from the group consisting of I2, I3-, I2SCN-, and I(SCN)2-.

In other aspects, the H2O2-producing enzyme and FRP enzyme are immobilized on hematite. In other aspects, the reaction container is a packed bed reactor. In other aspects, the reaction solution is pumped with a high-pressure pump through said packed bed reactor.

### BRIEF DESCRIPTION OF THE DRAWINGS

FIG. 1A shows the glucose oxidase (GOx) system for producing reactive oxidative species that inhibit or kill microbial pathogens.

FIG. 1B shows the glucose oxidase (GOx)/lactoperoxidase (LP) system for producing reactive oxidative species that inhibit or kill microbial pathogens.

FIG. 2A. Process flow diagram depicting the in-flow enzyme immobilization process on scaffold materials inside a packed bed reactor using recirculation.

FIG. 2B. Process flow diagram that produces an enzyme-free biocidal inducer of SAR. A pressurized biocatalytic packed bed flow reactor uses the two-enzyme system of glucose oxidase and lactoperoxidase along with a feed comprising about 35 mM KI, 8 mM KSCN, 300 mM glucose and 200 mM sodium acetate at pH 5.5.

FIG. 3. Activation of Systemic Acquired Resistance (SAR) in Tomato with and without Lactoperoxidase. 70/16 KI:KSCN 40/50 GOX:LP and 70/16 KI:KSCN 40U GOX was applied to tomato plants and gene expression was measured. Two days post application samples were taken and analyzed for expression of the marker gene pathogenesis realated-1 (PR1). It is a marker of activation of Systemic Acquired Resistance (SAR) in plants. Leaves sprayed with water were used as controls.

FIG. 4. Activation of SAR in tomato. 70/16 KI:KSCN 40/50 GOX:LP was applied to tomato plants and gene expression measured. Six days post application samples were taken and analyzed for expression of PR1. Leaves sprayed with water were used as controls.

FIG. 5. Activation of SAR in Potato without lactoperoxidase. 70/16 KI:KSCN 40/50 GOX:LP and 70/16 KI:KSCN 40U GOX were applied to potato plants and gene expression was measured. Two days post application, samples were taken and analyzed for expression of PRI expression. Leaves were sprayed with water were used as controls.

FIG. 6. Activation of SAR in Cabbage. Commercial adjuvant Li700, 40/50 GOX:LP+Li700, 35/8 KI:KSCN+Li700, 18/4 KI:KSCN 40/50 GOX:LP+Li700, 35/8 KI:KSCN 40/50 GOX:LP+Li700 were applied to cabbage plants and gene expression was measured. Three days post application, samples were taken and analyzed for expression of PR1 expression. Leaves sprayed with water were used as controls.

FIG. 7. Activation of SAR in Cucumber. 35/8 KI:KSCN 40/50 GOX:LP was applied to cucumber plants and gene expression measured. Six days post application samples were taken and analyzed for expression of PRI expression. Leaves sprayed with water were used as controls.

FIG. 8. Activation of SAR in Corn. 35/8 KI:KSCN 40/50 GOX:LP was applied to corn plants and gene expression was measured. Two days post application, samples were taken and analyzed for expression of PRI expression. Leaves sprayed with water were used as controls.

FIG. 9. Activation of SAR in Corn. 35/8 KI:KSCN 40/50 GOX:LP was applied to wheat plants and gene expression was measured. Four days post application, samples were taken and analyzed for PRI expression. Leaves sprayed with water were used as controls.

FIG. 10. List of primers used for qPCR experiments to query activation of Systemic Required Resistance.

FIG. 11. Percent final foliar black rot disease on cabbage plants inoculated in the field with X. campestris pv campestris. Treatments with the same letter are not significantly different.

FIG. 12. Percent final foliar downy mildew disease on cucumber plants naturally infected in the field with Pseudoperonospora cubensis. Treatments with the same letter are not significantly different.

FIG. 13. Percent final foliar powdery mildew disease on pumpkin plants naturally infected in the field with Podosphaera xanthii. Treatments with the same letter are not significantly different.

FIG. 14. Iodide content in tomato fruit after field application of AX formulations.

FIG. 15. Iodide content in tomato (TL) and potato leaves (PL) after application of AX formulations.

FIG. 16A. Fungal disease eradicant activity on corn salad seeds. Full formulation parameters displayed from left to right. A) 35 mM KI, 8 mM KSCN, 300 mM Glucose, 200 mM Sodium Acetate, 20 U/mL Glucose-Oxidase, 25 U/mL Lactoperoxidase. B) 35 mM KI, 4 mM KSCN, 300 mM Glucose, 200 mM Sodium Acetate, 20 U/mL Glucose-Oxidase, 25 U/mL Lactoperoxidase, C) 35 mM KI, 0 mM KSCN, 300 mM Glucose, 200 mM Sodium Acetate, 20 U/mL Glucose-Oxidase, 25 U/mL Lactoperoxidase, D) 35 mM KI, 8 mM KSCN, 150 mM Glucose, 200 mM Sodium Acetate, 20 U/mL Glucose-Oxidase, 25 U/mL Lactoperoxidase, E) 35 mM KI, 8 mM KSCN, 75 mM Glucose, 200 mM Sodium Acetate, 20 U/mL Glucose-Oxidase, 25 U/mL Lactoperoxidase.

FIG. 16B. Bacterial disease eradicant activity on corn salad seeds. Full formulation parameters displayed from left to right. A) 35 mM KI, 8 mM KSCN, 300 mM Glucose, 200 mM Sodium Acetate, 20 U/mL Glucose-Oxidase, 25 U/mL Lactoperoxidase. B) 35 mM KI, 4 mM KSCN, 300 mM Glucose, 200 mM Sodium Acetate, 20 U/mL Glucose-Oxidase, 25 U/mL Lactoperoxidase, C) 35 mM KI, 0 mM KSCN, 300 mM Glucose, 200 mM Sodium Acetate, 20 U/mL Glucose-Oxidase, 25 U/mL Lactoperoxidase, D) 35 mM KI, 8 mM KSCN, 150 mM Glucose, 200 mM Sodium Acetate, 20 U/mL Glucose-Oxidase, 25 U/mL Lactoperoxidase, E) 35 mM KI, 8 mM KSCN, 75 mM Glucose, 200 mM Sodium Acetate, 20 U/mL Glucose-Oxidase, 25 U/mL Lactoperoxidase.

FIG. 17. Bacterial growth inhibition in nutrient broth. Full formulation parameters displayed from left to right. 1) 35 mM KI, 8 mM KSCN, 300 mM Glucose, 200 mM Sodium Acetate, 20 U/mL Glucose-Oxidase, 25 U/mL Lactoperoxidase. 2) 35 mM KI, 4 mM KSCN, 300 mM Glucose, 200 mM Sodium Acetate, 20 U/mL Glucose-Oxidase, 25 U/mL Lactoperoxidase, 3) 35 mM KI, 0 mM KSCN, 300 mM Glucose, 200 mM Sodium Acetate, 20 U/mL Glucose-Oxidase, 25 U/mL Lactoperoxidase

FIG. 18. Fungal growth inhibition on media plates. Full formulation parameters displayed from left to right. 1) 35 mM KI, 8 mM KSCN, 300 mM Glucose, 200 mM Sodium Acetate, 20 U/mL Glucose-Oxidase, 25 U/mL Lactoperoxidase. 2) 35 mM KI, 2 mM KSCN, 300 mM Glucose, 200 mM Sodium Acetate, 20 U/mL Glucose-Oxidase, 25 U/mL Lactoperoxidase, 3) 35 mM KI, 0 mM KSCN, 300 mM Glucose, 200 mM Sodium Acetate, 20 U/mL Glucose-Oxidase, 25 U/mL Lactoperoxidase.

FIG. 19. List of primers used for qPCR analyses that monitored Systemic Acquired Resistance (SAR), flowering and fruit ripening gene activation.

FIG. 20. Nonexpressor of PR Genes 1 (NPR1) activation. AX formulations having 2 mM KI and 0.2 mM KI with 5/6 U/ml GOx/LP, and 2/3 U/ml GOx/LP respectively, were applied to tomato plants and gene expression was measured. One day post application, samples were taken and analyzed for NPR1 expression as a marker for SAR activation. Water was used for controls.

FIG. 21. Pathogenesis-Related 1 (PR1), a gene involved in SAR that is downstream of NPR1, is significantly upregulated by 2 mM KI and 0 mM KSCN AX treatment compared to water 24 hours post foliar application. AX formulations consisting of 2 mM KI and 0.2 mM KI with 5/6 U/ml GOx/LP, and 2/3 U/ml GOx/LP respectively, were applied to tomato plants and gene expression was measured. One day post application samples were taken and analyzed for PRI expression, a marker for SAR activation in plants. Water was used for controls.

FIG. 22. Salicylic Acid Methyltransferase 1 (SAMT1), a gene involved in the metabolism of Salicylic Acid that elicits SAR is significantly upregulated in tomato by the 2/0 AX treatment (i.e. 2 mM KI, 0 mM KSCN) but not the 0.2/0 (i.e. 0.2 mM KI, 0 mM KSCN) treatment compared to water 24 hours post foliar application. AX formulations having 2 mM KI and 0.2 mM KI with 5/6 U/ml GOx/LP and 2/3 U/ml GOx/LP, respectively, were applied to tomato plants and gene expression was measured. One day post application, samples were taken and analyzed for expression of SAMT1, a marker gene for SASR activation. Water was used for controls.

FIG. 23. NPR was upregulated by the 4/0.8 and 4/0.8+no LP treatment when compared to water 24 hours post foliar application. AX formulations having 4/0.8 mM KI/KSCN with and without lactoperoxidase at 5/6 and 5/0 U/ml GOx/LP, respectively, were applied to cucumber plants and gene expression measured. One day post application, samples were taken and analyzed for NPR1 expression. Water was used for controls.

FIG. 24. PR1, a gene involved in SAR in cucumber and downstream of NPR1, was upregulated by the 4/0.8 and 4/0.8+no LP treatment when compared to water 24 hours post foliar application. AX formulations having 4/0.8 mM KI/KSCN with and without lactoperoxidase at 5/6 and 5/0 U/ml GOx/LP, respectively, were applied to cucumber plants and gene expression measured. One day post application, samples were taken and analyzed for PRI expression. Water wa used as controls.

FIG. 25. PR5, a gene involved in SAR and downstream of NPR1, is upregulated by the 4/0.8+No LP treatment when compared to water. AX formulations having 4/0.8 mM KI/KSCN with and without lactoperoxidase at 5/6 and 5/0 U/ml GOx/LP, respectively, were applied to cucumber plants and gene expression was measured. One day post application, samples were taken and analyzed for PR5 expression. Water was used for controls.

FIG. 26. Efficacy of AX 4/0.8 mM KI/KSCN foliar applications (with and without LP at 5/6 and 5/0 U/ml GOx/LP, respectively) were compared to commercial and non-treated controls for bacterial (Pseudomonas syringae pv. aptata) leaf spot (BLS) management in table beets. Final disease severity was calculated as area under disease progress curve (AUDPC). Treatments with the same letter were not significantly different using Tukey's HSD multiple comparison of means 0.05. AX formulations were applied folliarly at 4/0.8 mM KI/KSCN with and without LP at 5/6 and 5/0 U/ml GOx/LP.

FIG. 27. Efficacy of AX 4/0.8 mM KI/KSCN foliar applications (with and without LP at 5/6 and 5/0 U/ml GOx/LP, respectively) were compared with commercial pesticides and non-treated controls for Cercospora (Cercospora beticola) leaf spot (CLS) management in table beets. Final disease severity was calculated as AUDPC. Treatments with the same letter were not significantly different using Tukey's HSD multiple comparison of means 0.05. AX foliar applications of 4/0.8 mM KI/KSCN with and without LP at 5/6 and 5/0 U/ml GOx/LP were used.

FIG. 28. Efficacy of AX foliar applications, with and without LP and KSCN for managing Powdery Mildew (Oidium lycopersicum) in tomato compared to commercial pesticides and non-treated controls. Disease severity was calculated as final AUDPC. Treatments with the same letter were not significantly different by Tukey's HSD multiple comparison of means 0.05. AX foliar applications of 4/0.8 mM KI/KSCN+10/12 U/ml GOx/LP, 4/0 mM KI/KSCN+10/12 U/ml GOx/LP, and 4/0.8 mM KI/KSCN+10/0 U/ml GOx/LP were used.

FIG. 29. Efficacy of AX foliar applications with and without KSCN for Downy Mildew (Pseudoperonospra cubensis) management in cucumber were compared to commercial pesticides and non-treated controls. Disease severity was calculated as final AUDPC. Treatments with the same letter were not significantly different using Tukey's HSD multiple comparison of means 0.05. AX foliar applications of 4/0.8 mM KI/KSCN+10/12 U/ml GOx/LP, 4/0 mM KI/KSCN+10/12 U/ml GOx/LP, commercial pesticides, and non-treated controls were used.

FIG. 30. Efficacy of AX foliar applications, with and without LP and KSCN, for managing Early Blight (Alternaria solani) in tomato were compared to commercial pesticides and non-treated controls. Data shown as final % disease. Treatments with the same letter were not significantly different using Tukey's HSD multiple comparison of means 0.05. AX foliar applications of 4/0.8 mM KI/KSCN+10/12 U/ml GOx/LP, 4/0 mM KI/KSCN+10/12 U/ml GOx/LP, 4/0.8 mM KI/KSCN+10/0 U/ml GOx/LP, commercial pesticides, and non-treated controls were used.

FIG. 31. Gibberellic Acid Stimulated Transcript 1 (GAST1), a gene involved in regulation of flowering in tomato, is upregulated by the 2/0 AX treatment 24 hours post foliar application when compared to water. Application of either 2 mM KI or 0.2 mM KI along with 5/6 U/ml GOx/LP and 2/3 U/ml GOx/LP, respectively, were applied to tomato plants and gene expression measured. One day post application samples were taken and analyzed for GASTI expression. Water was used as controls.

FIG. 32. Fruitfull-Like 2 (FUL2), a gene that regulates flowering time and fruit development in tomato, is upregulated by the 2/0 AX treatment 24 hours post foliar application when compared to water. Application of either 2 mM KI or 0.2 mM KI along with 5/6 U/ml GOx/LP and 2/3 U/ml GOx/LP, respectively, were applied to tomato plants and gene expression measured. One day post application samples were taken and analyzed for FUL2 expression. Water was used as controls.

FIG. 33. Gretchen Hagen 3.24 (GH3.24), a gene involved in tomato fruit ripening and quality, is upregulated by the 2/0 AX treatment 24 hours post foliar application when compared to water. Application of either 2 mM KI or 0.2 mM KI along with 5/6 U/ml GOx/LP and 2/3 U/ml GOx/LP, respectively, were applied to tomato plants and gene expression measured. One day post application samples were taken and analyzed for GH3.24 expression. Water was used as controls.

FIG. 34. FIG. 34. Total tomato fruit yield (Kg) after AX formulation treatments of 2 mM KI or 0.2 mM KI along with 5 U/ml GOx, 6 U/ml LP and 2/3 U/ml GOx/LP respectively. Six tomato plants per treatment received weekly 1 ml foliar spray applications approximately three weeks after planting. Approximately 11 weeks after planting, the plants received weekly 2 mL foliar spray applications until approximately 14 weeks after harvest. Tomato harvest began approximately at week 15 and went through approximately week 22. The water, 0.2 mM KI+Gox & LP, and the 2 mM KI+GOx & LP, yielded 23.1 Kg, 31.8 Kg, and 30.6 Kg of tomato fruit, respectively.

### DETAILED DESCRIPTION OF THE INVENTION

The present invention provides compositions and methods for reducing microbial, nematodal, and oomycotic contamination or infection in plants and products therefrom. In particular, it has been surprisingly found that certain chemoenzymatic formulations may be optimized for eliciting a Systemic Acquired Resistance (SAR) in plants. Furthermore, it was surprisingly found that certain chemoenzymatic formulations act as a biostimulant for plants. Other chemoenzymatic formulations kill undesired microbes.

SAR is a mechanism of induced plant defense that confers long-lasting protection against a broad spectrum of microorganisms. An initial infection on a lower leaf with a fungal, bacterial, or viral pathogen that is recognized by the plant leads to a long-lasting and broad protection against fungal, oomycete, bacterial, or viral pathogens in distal leaves and independently of the nature of the first invader. SAR requires the signal molecule salicylic acid (SA) which causes the accumulation of pathogenesis-related proteins that contribute to resistance. The isochorismate pathway is the major source of SA during SAR. In response to SA, the positive regulator protein Nonexpresser of PR gene1 (NPR1) moves to the nucleus where it interacts with TGA transcription factors to induce defense gene expression. This activates SAR. Durrant and Dong, Annu Rev Phytopathol. 42:185-209. Doi: 10.1146/annurev.phyto.42.040803.140421. PMID: 15283665 (2004), incorporated by reference herein in its entirety.

The invention provides foliar crop protection products that are biocatalytically generated by enzymes (referred to as AX). They have a unique dual mode of action comprising (i) broad-spectrum biocidal action and (ii) induction of systemic acquired response (SAR) in plants. This biopesticide comprises iodo, hypo-, and inter-pseudo-halogen species that are oxidative products biocatalytically generated from thiocyanate and iodide by glucose oxidase (GOx). In some embodiments, the Gox is combined with lactoperoxidase (LP). GOx generates hydrogen peroxide (H₂O2) from D-glucose and oxygen, while LP catalyzes the oxidation of thiocyanate and iodide ions with H₂O₂ to transiently produce hypothiocyanite (OSCN-) and hypoiodite (OI-). These are subsequently converted to a mixture of oxidative species including iodine (I2), triiodide (I3-) and interspecies such as I₂SCN- and I(SCN)₂—. In some embodiments, the compositions are dormant and become active upon exposure to hydration, oxygen, or mixing.

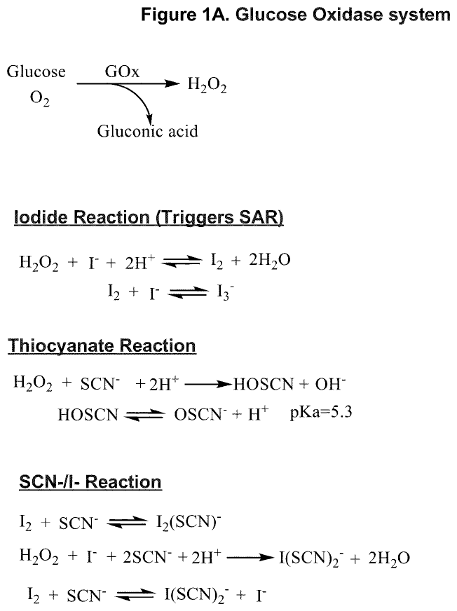

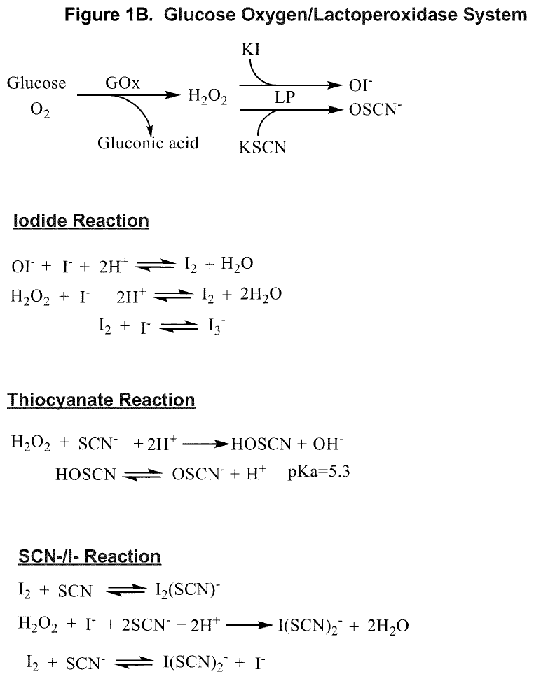

The present invention provides compositions and methods for reducing microbial contamination or infection in plants. This is accomplished, for the first time, by optimizing the SAR in plants utilizing particular formulations of glucose, glucose oxidase, an iodide source (such as KI), and a thiocyanate source (such as KSCN). Glucose oxidase is a hydrogen peroxide-producing (HPP) enzyme. In some embodiments the glucose/glucose oxidase system is substituted for another HPP enzyme. FIG. 1A. In other embodiments, the optimized system further comprises a free radical-producing enzyme (FRP) such as lactoperoxidase. FIG. 1B.

Additionally, the inventors have made the surprising discovery that, in fact, SAR can be induced by formulations having just an iodide source and a hydrogen peroxide source. Such compositions surprisingly may lack LP or a thiocyanate source. In particular, it was surprisingly found that at concentrations where topical hydrogen peroxide would not normally induce SAR, certain concentrations of iodide salt in combination with hydrogen peroxide at those concentrations now triggers SAR. Thus, the invention provides, for the first time, a method of inducing microbial resistance in plants with a composition that has, at its core, KI, GOx, and glucose, or another hydrogen peroxide source. In a preferred embodiment, the composition comprises about 2 mM KI, 5 U/ml GOx, and 50 mM glucose. In other embodiments, the composition might further comprise LP, and in preferred embodiments, 6 U/ml LP.

Without being bound by theory, the hydrogen peroxide source in the compositions and methods of the invention mediates the oxidation of iodide into reactive species (e.g. I⁻, IO⁻, IO²⁻, etc.) which then passes through into the leaves and exposes the plant cells to trigger SAR. In the prior art, hydrogen peroxide triggers SAR by a different pathway—but only when physically injected into the leaves. (See, e.g., Alvarez et al., Cell 92:773-784 (1998), incorporated by reference herein in its entirety. Without the hydrogen peroxide, a much larger amount of iodide is required to get appreciable SAR activation. Thus, the invention provides, for the first time, compositions and methods that induce SAR with much lower iodide amounts that is sprayed directly onto the leaves due to the hydrogen peroxide-mediated oxygenation of iodide in situ.

Additionally, the invention provides the important advance of lowering the cost of materials, being more sustainable, and reducing plant phytotoxicity. This is accomplished by optimizing the iodide and peroxide source in the compositions and methods. It is also surprisingly accomplished in some embodiments by omitting lactoperoxidase and/or thiocyanates. It is well-known that peroxides and free radicals (e.g. thiocyanates) are phytotoxic. Additionally, too much iodide salt is unfavorable for the soil because it introduces too much salt.

The inventors have also surprisingly discovered that in certain plants (e.g. tomatoes), certain compositions of the invention comprising KI, GOx, LP, and glucose act as a biostimulant. Such biostimulation is caused by early induction of flowering genes that result in higher fruit yields. In a preferred embodiment, the composition comprises about 2 mM KI, 5 U/ml GOx, 6 U/ml LP, and 50 mM glucose.

The inventors have also surprisingly discovered that certain compositions of the invention comprising KI, a thiocyanate, GOx, and glucose are biocidal. In a preferred embodiment, the composition comprises about 4 mM KI, 0.8 mM KSCN, 10 U/ml GOx, 12 U/ml LP, and 50 mM Glucose.

In other embodiments, the enzymes are magnetically-entrapped in the mesopores of self-assembled magnetic nanoparticles to form bionanocatalysts (BNC). Self-assembly involves the magnetic attraction between individual nanoparticles to form the mesoporous BNCs in which the enzymes are magnetically entrapped. These magnetically-immobilized enzymes may be in solid or liquid compositions that are stable or inactive. Thus, they may be stored prior to or after incorporation into products.

When the fungicidal activities are required, these multicomponent compositions are activated by mixing, hydration, and/or exposure to oxygen. The HPP enzyme (e.g. Glucose oxidase) acts on substrates to produce hydrogen peroxide and, e.g. D-glucono-δ-lactone. The FRP (e.g. lactoperoxidase) enzyme acts on the hydrogen peroxide and one or more further substrates to produce free radicals. The hydrogen peroxide and free radicals have antimicrobial properties. In alternative embodiments, hydrogen peroxide is provided as opposed to a hydrogen peroxide producing enzyme plus its substrates. The antimicrobial activities may be dormant and activated by exposure to hydration and/or oxygen. The disclosures of Int'l Pub. Nos. WO2012122437, WO2014055853, WO2016186879, and WO2018034877 are incorporated by reference herein in their entirety.

In some embodiments, the enzymes are magnetically-entrapped within the mesopores of Self-assembled mesoporous aggregates of magnetic nanoparticles (MNPs). MNPs comprising magnetically-entrapped peroxidases are highly active and robust. The technology is a powerful blend of biochemistry, nanotechnology, and bioengineering at three integrated levels of organization: Level 1 is the self-assembly of peroxidase and oxidase enzymes with magnetic nanoparticles (MNP) for the synthesis of magnetic mesoporous nanoclusters. This level uses a mechanism of molecular self-entrapment to immobilize and stabilize enzymes. Level 2 is the stabilization of the MNPs into other matrices. Level 3 is product conditioning and packaging for Level 1+2 delivery. The assembly of magnetic nanoparticles adsorbed to enzyme is herein also referred to as a “bionanocatalyst” (BNC).

MNP immobilization provides highly active and cost-effective peroxidases. Peroxidases are very potent enzymes yet notoriously difficult to deploy in industrial settings due to strong inhibition in presence of excess peroxide. NPs increase peroxidation activity and reduce their inhibition which renders them industrially useful. Additionally, the MNPs allow for a broader range of operating conditions such as temperature, ionic strength and pH. The size and magnetization of the MNPs affect the formation and structure of the NPs, all of which have a significant impact on the activity of the entrapped enzymes. By virtue of their surprising resilience under various reaction conditions, MNPs can be used as improved enzymatic or catalytic agents where other such agents are currently used. Furthermore, they can be used in other applications where enzymes have not yet been considered or found applicable.

The BNC contains mesopores that are interstitial spaces between the magnetic nanoparticles. The enzymes are preferably embedded or immobilized within at least a portion of mesopores of the BNC. As used herein, the term “magnetic” encompasses all types of useful magnetic characteristics, including permanent magnetic, superparamagnetic, paramagnetic, ferromagnetic, and ferrimagnetic behaviors.

The magnetic nanoparticle or BNC has a size in the nanoscale, i.e., generally no more than 500 nm. As used herein, the term “size” can refer to a diameter of the magnetic nanoparticle when the magnetic nanoparticle is approximately or substantially spherical. In a case where the magnetic nanoparticle is not approximately or substantially spherical (e.g., substantially ovoid or irregular), the term “size” can refer to either the longest the dimension or an average of the three dimensions of the magnetic nanoparticle. The term “size” may also refer to an average of sizes over a population of magnetic nanoparticles (i.e., “average size”).

In different embodiments, the magnetic nanoparticle has a size of precisely, about, up to, or less than, for example, 500 nm, 400 nm, 300 nm, 200 nm, 100 nm, 50 nm, 40 nm, 30 nm, 25 nm, 20 nm, 15 nm, 10 nm, 5 nm, 4 nm, 3 nm, 2 nm, or 1 nm, or a size within a range bounded by any two of the foregoing exemplary sizes.

In the BNC, the individual magnetic nanoparticles can be considered to be primary nanoparticles (i.e., primary crystallites) having any of the sizes provided above. The aggregates of nanoparticles in a BNC are larger in size than the nanoparticles and generally have a size (i.e., secondary size) of at least about 5 nm. In different embodiments, the aggregates have a size of precisely, about, at least, above, up to, or less than, for example, 5 nm, 8 nm, 10 nm, 12 nm, 15 nm, 20 nm, 25 nm, 30 nm, 35 nm, 40 nm, 45 nm, 50 nm, 60 nm, 70 nm, 80 nm, 90 nm, 100 nm, 150 nm, 200 nm, 300 nm, 400 nm, 500 nm, 600 nm, 700 nm, or 800 nm, or a size within a range bounded by any two of the foregoing exemplary sizes.

Typically, the primary and/or aggregated magnetic nanoparticles or BNCs thereof have a distribution of sizes, i.e., they are generally dispersed in size, either narrowly or broadly dispersed. In different embodiments, any range of primary or aggregate sizes can constitute a major or minor proportion of the total range of primary or aggregate sizes. For example, in some embodiments, a particular range of primary particle sizes (for example, at least about 1, 2, 3, 5, or 10 nm and up to about 15, 20, 25, 30, 35, 40, 45, or 50 nm) or a particular range of aggregate particle sizes (for example, at least about 5, 10, 15, or 20 nm and up to about 50, 100, 150, 200, 250, or 300 nm) constitutes at least or above about 50%, 60%, 70%, 80%, 90%, 95%, 98%, 99%, or 100% of the total range of primary particle sizes. In other embodiments, a particular range of primary particle sizes (for example, less than about 1, 2, 3, 5, or 10 nm, or above about 15, 20, 25, 30, 35, 40, 45, or 50 nm) or a particular range of aggregate particle sizes (for example, less than about 20, 10, or 5 nm, or above about 25, 50, 100, 150, 200, 250, or 300 nm) constitutes no more than or less than about 50%, 40%, 30%, 20%, 10%, 5%, 2%, 1%, 0.5%, or 0.1% of the total range of primary particle sizes.

The aggregates of magnetic nanoparticles (i.e., “aggregates”) or BNCs thereof can have any degree of porosity, including a substantial lack of porosity depending upon the quantity of individual primary crystallites they are made of. In particular embodiments, the aggregates are mesoporous by containing interstitial mesopores (i.e., mesopores located between primar magnetic nanoparticles, formed by packing arrangements). The mesopores are generally at least 2 nm and up to 50 nm in size. In different embodiments, the mesopores can have a pore size of precisely or about, for example, 2, 3, 4, 5, 10, 12, 15, 20, 25, 30, 35, 40, 45, or 50 nm, or a pore size within a range bounded by any two of the foregoing exemplary pore sizes. Similar to the case of particle sizes, the mesopores typically have a distribution of sizes, i.e., they are generally dispersed in size, either narrowly or broadly dispersed. In different embodiments, any range of mesopore sizes can constitute a major or minor proportion of the total range of mesopore sizes or of the total pore volume. For example, in some embodiments, a particular range of mesopore sizes (for example, at least about 2, 3, or 5, and up to 8, 10, 15, 20, 25, or 30 nm) constitutes at least or above about 50%, 60%, 70%, 80%, 90%, 95%, 98%, 99%, or 100% of the total range of mesopore sizes or of the total pore volume. In other embodiments, a particular range of mesopore sizes (for example, less than about 2, 3, 4, or 5 nm, or above about 10, 15, 20, 25, 30, 35, 40, 45, or 50 nm) constitutes no more than or less than about 50%, 40%, 30%, 20%, 10%, 5%, 2%, 1%, 0.5%, or 0.1% of the total range of mesopore sizes or of the total pore volume.

The magnetic nanoparticles can have any of the compositions known in the art. In some embodiments, the magnetic nanoparticles are or include a zerovalent metallic portion that is magnetic. Some examples of such zerovalent metals include cobalt, nickel, and iron, and their mixtures and alloys. In other embodiments, the magnetic nanoparticles are or include an oxide of a magnetic metal, such as an oxide of cobalt, nickel, or iron, or a mixture thereof. In some embodiments, the magnetic nanoparticles possess distinct core and surface portions. For example, the magnetic nanoparticles may have a core portion composed of elemental iron, cobalt, or nickel and a surface portion composed of a passivating layer, such as a metal oxide or a noble metal coating, such as a layer of gold, platinum, palladium, or silver. In other embodiments, metal oxide magnetic nanoparticles or aggregates thereof are coated with a layer of a noble metal coating. The noble metal coating may, for example, reduce the number of charges on the magnetic nanoparticle surface, which may beneficially increase dispersibility in solution and better control the size of the BNCs. The noble metal coating protects the magnetic nanoparticles against oxidation, solubilization by leaching or by chelation when chelating organic acids, such as citrate, malonate, or tartrate, are used in the biochemical reactions or processes. The passivating layer can have any suitable thickness, and particularly, at least, up to, or less than, about for example, 0.1 nm, 0.2 nm, 0.3 nm, 0.4 nm, 0.5 nm, 0.6 nm, 0.7 nm, 0.8 nm, 0.9 nm, 1 nm, 2 nm, 3 nm, 4 nm, 5 nm, 6 nm, 7 nm, 8 nm, 9 nm, or 10 nm, or a thickness in a range bounded by any two of these values.

Magnetic materials useful for the invention are well-known in the art. Non-limiting examples comprise ferromagnetic and ferromagnetic materials including ores such as iron ore (magnetite or lodestone), cobalt, and nickel. In other embodiments, rare earth magnets are used. Non-limiting examples include neodymium, gadolinium, sysprosium, samarium-cobalt, neodymium-iron-boron, and the like. In yet further embodiments, the magnets comprise composite materials. Non-limiting examples include ceramic, ferrite, and alnico magnets. In preferred embodiments, the magnetic nanoparticles have an iron oxide composition. The iron oxide composition can be any of the magnetic or superparamagnetic iron oxide compositions known in the art, e.g., magnetite (FesO/O, hematite (α-Fe2θ 3), maghemite (γ-Fe2C>3), or a spinel ferrite according to the formula AB₂O₄, wherein A is a divalent metal (e.g., Xn²⁺, Ni²⁺, Mn²⁺, Co²⁺, Ba²⁺, Sr²⁺, or combination thereof) and B is a trivalent metal (e.g., Fe³⁺, Cr³⁺, or combination thereof).

The individual magnetic nanoparticles or aggregates thereof or BNCs thereof possess any suitable degree of magnetism. For example, the magnetic nanoparticles, BNCs, or BNC scaffold assemblies can possess a saturated magnetization (Ms) of at least or up to about 5, 10, 15, 20, 25, 30, 40, 45, 50, 60, 70, 80, 90, or 100 emu/g. The magnetic nanoparticles, BNCs, or BNC-scaffold assemblies preferably possess a remanent magnetization (Mr) of no more than (i.e., up to) or less than 5 emu/g, and more preferably, up to or less than 4 emu/g, 3 emu/g, 2 emu/g, 1 emu/g, 0.5 emu/g, or 0.1 emu/g. The surface magnetic field of the magnetic nanoparticles, BNCs, or BNC-scaffold assemblies can be about or at least, for example, about 0.5, 1, 5, 10, 50, 100, 200, 300, 400, 500, 600, 700, 800, 900, or 1000 Gauss (G), or a magnetic field within a range bounded by any two of the foregoing values. If microparticles are included, the microparticles may also possess any of the above magnetic strengths.

The magnetic nanoparticles or aggregates thereof can be made to adsorb a suitable amount of enzyme, up to or below a saturation level, depending on the application, to produce the resulting BNC. In different embodiments, the magnetic nanoparticles or aggregates thereof may adsorb about, at least, up to, or less than, for example, 1, 5, 10, 15, 20, 25, or 30 μmol/m2 of enzyme. Alternatively, the magnetic nanoparticles or aggregates thereof may adsorb an amount of enzyme that is about, at least, up to, or less than, for example, about 10%, 20%, 30%, 40%, 50%, 60%, 70%, 80%, 90%, or 100% of a saturation level.

The magnetic nanoparticles or aggregates thereof or BNCs thereof possess any suitable pore volume. For example, the magnetic nanoparticles or aggregates thereof can possess a pore volume of about, at least, up to, or less than, for example, about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or

1 cm³/g, or a pore volume within a range bounded by any two of the foregoing values. The magnetic nanoparticles or aggregates thereof or BNCs thereof possess any suitable specific surface area. For example, the magnetic nanoparticles or aggregates thereof can have a specific surface area of about, at least, up to, or less than, for example, about 50, 60, 70, 80, 90, 100, 110, 120, 130, 140, 150, 160, 170, 180, 190, or 200 m 2/g.

MNPs, their structures, organizations, suitable enzymes, and uses are described in WO2012122437 and WO2014055853, incorporated by reference herein in their entirety.

The compositions and methods of the invention, among other things, reduce or eliminate plant death due to pathogens. In some embodiments, the invention reduces or eliminates “damping off.” The American Phytopathological Society defines damping-off as “the death of a seedling before or shortly after emergence due to decomposition of the root and/or lower stem; it is common to distinguish between pre-emergence damping-off and post-emergence damping-off. Pre-emergence damping-off occurs before a seedling emerges from the soil line. Post-emergence damping-off occurs shortly after a seedling emerges from the soil line. The disease is commonly caused by the fungus Rhizoconia solani and numerous species in the oomycete genus Pythium, although other fungi and oomycetes can contribute. The disease is not crop-specific and causes losses to all agricultural crops.

In some embodiments, the invention provides a two-enzyme, biocatalytically generated foliar crop protection product with a unique dual mode of action comprising (i) broad-spectrum biocidal action and (ii) induction of systemic acquired response (SAR) in plants. In addition to a dual mode of action, the iodine-based foliar or seed product results in iodine accumulation in plants thus acting as a biofortification agent that stimulates plant growth and increases crop yield. This biopesticide consists of iodo, hypo-, and inter-pseudo-halogen species that are oxidative products may be biocatalytically generated. In some embodiments they are generated from thiocyanate and iodide. In other embodiments they are generated by two enzymes: glucose oxidase (GOx) and lactoperoxidase (LP). GOx generates hydrogen peroxide (H₂O2) from D-glucose and oxygen, while LP catalyzes the oxidation of thiocyanate and iodide ions with H₂O₂ to transiently produce hypothiocyanite (OSCN-) and hypoiodite (OI⁻). These are subsequently converted to a mixture of oxidative species including iodine (I₂), triiodide (I₃⁻) and interspecies such as ISCN⁻ and I(SCN)₂⁻

In some embodiments, the AX biopesticide is produced by a process wherein the solid chemical precursors and lyophilized enzymes are reconstituted in water using industry standard tank mixers. In some embodiments, they are mixed the day prior or day of foliar application. Thus, in some embodiments, upon reconstitution, the solution is stirred overnight and sprayed with standard equipment. During spraying, exposure to oxygen reignites the enzymatic cascade and effects in situ production of biocidal species on leaves.

In other embodiments, formulations have antimicrobial properties against a wide array of pathogens. In some embodiments, the pathogens include pathogenic plant bacteria species such as Acidovorax avenae, Agrobacterium tumefaciens, Burkholderia andropogonis, Burkholderia caryophylli, Burkholderia glumae, Candidatus Liberibacter, Candidatus Phytoplasma solani, Clavibacter michiganensis, Dickeya dadantii, Erwinia psidii, Pectobacterium atrosepticum, Pectobacterium betavasculorum, Pectobacterium carotovorum, Pectobacterium carotovorum subsp. betavasculorum, Pectobacterium wasabiae, Phytoplasma, Pseudomonas amygdali, Pseudomonas asplenii, Pseudomonas caricapapayae, Pseudomonas cichorii, Pseudomonas coronafaciens, Pseudomonas corrugate, Pseudomonas ficuserectae, Pseudomonas flavescens, Pseudomonas fuscovaginae, Pseudomonas helianthi, Pseudomonas marginalis, Pseudomonas oryzihabitans, Pseudomonas palleroniana, Pseudomonas papaveris, Pseudomonas salomonii, Pseudomonas savastanoi, Pseudomonas syringae, Pseudomonas tomato, Pseudomonas turbinellae, Pseudomonas viridiflava, Psyllid yellows, Ralstonia solanacearum, Rhodococcus fascians, Spiroplasma citri, Xanthomonas axonopodis, Xanthomonas campestris, Xanthomonas oryzae, Xylella fastidiosa, Escherichia coli, Salmonella enterica, Listeria monocytogenes, and other plant, animal, human, soilborne, and environmental pathogens.

In other embodiments, the formulations have antimicrobial properties against non-plant pathogen bacteria including Escherichia Coli, Brucella sp., Vibrio sp., Serrati asp., Nocardia sp., Leptospira sp., Mycobacterium sp., Clostridium sp., Bacillus sp., Pseudomonas sp. Staphylococcus sp., Neisseria sp., Haemophilus sp., Helicobacter sp., Mycoplasma sp., Pseudomonas sp. Treponema sp., and Yersinia sp.

In other embodiments, the fungicidal formulations are effective against plant pathogenic fungi including genera such as Alternaria sp., Armillaria sp. Ascochyta sp., Aspergillus sp., Bipoloaris, Bjerkandera sp., Botrytis sp., Ceratobasidium sp., Cercospora sp., Chrysimyxa sp., Cladosporium sp., Cochliobolus sp., Coleosporium sp., Colletotrichum sp., Cylindrocladium sp., Cytospora sp., Diaporthe sp., Didymella sp., Drechslera sp., Erysiphe sp, Exobasidium sp., Fusarium sp., Ganoderma sp., Gibberella sp., Gymnospragium sp., Helicobasidium sp., Inonotus sp., Leptosphaeria sp., Leucostoma sp. Marasmius sp., Microspaera sp., Mucor sp., Mycosphaerella sp., Nectria sp., Oidium sp., Passalora sp., Pestalotiopsis sp., Phaeoramularia sp., Phoma sp., Phyllosticta sp., Pseudocercospora sp., Puccinia sp., Pyrenophora sp., Rhizoctonia sp., Rhizopus sp., Septoria sp., Sphaceloma sp., Stemphylium sp., Stigmina sp., Tilletia sp., Typhula sp., Uromyces sp., Ustilago sp., and Verticillium sp.

In other embodiments, the fungicidal formulations are effective against plant pathogenic oomycetes including genera such as Aphanomyces sp., Bremia sp., Peronosclerospora sp., Peronospora sp., Phytophthora sp., Plasmopara sp., Pseudoperonospora sp., Pythium sp. and Sclerophthora sp. In preferred embodiments, the oomycetes are Phytophthora infestans, Hyaloperonospora arabidopsidis, Phytophthora ramorum, Phytophthora sojae, Phytophthora capsici, Plasmopara viticola, Phytophthora cinnamomi, Phytophthora parasitica, Pythium ultimum, or Albugo candida.

A number of genera and species of nematodes are highly damaging to a great range of plants, including foliage plants, agronomic and vegetable crops, fruit and nut trees, turfgrass, and forest trees. Thus, in some embodiments, the formulations of the invention are effective against nematodes such as Meloidogyne species (spp.), Heterodera spp., Globodera spp., Pratylenchus spp., Helicotylenchus spp., Radopholus similis, Ditylenchus dipsaci, Rotylenchulus reniformis, Xiphinema spp, Aphelenchoides spp., Toxocara spp., Bursaphelenchus xylophilus, and trichinella spiralis.

In other embodiments, the invention is effective against plant viruses that include plant viruses such as Mosaic Viruses, Mottle Viruses, Begomoviruses, Carlaviruses, Carmoviruses, Criniviruses, Fabaviruses, Furoviruses, Machlomoviruses, Macluraviruses, Necroviruses, Potexviruses, Tenuiviruses, and Tospoviruses.

In some embodiments of the invention, AX acts as a plant biostimulant by increasing iodine levels. The plants photosynthetic machinery is also affected as many iodinated proteins are involved in photosynthesis (Kiferle et al., Front. Plant Sci. 12 (616868): (2021)))

Iodine is a beneficial plant nutrient and can cause early flowering and increase yield and as such our formulations may as well (Kiferle et al., Sci. Rep. 12 (14655): (2022)). AX induces plant defense genes and activates other signaling pathways such as the abscisic acid pathway. This attenuates plant responses to environmental stresses. The foregoing references are incorporated by reference in their entirety.

Iodine increases the antioxidant response of plants, alters gene expression, and causes protein iodination (Medrano-Macías et al., Front. Plant Sci. 7 (1146): (2016); Kiferle et al., Front. Plant Sci. 12 (616868): (2021)). Notably, the effect of iodine on the antioxidant potential of plants depends on the source of iodine (KI vs. KIO₃) and the plant species (reviewed in (Medrano-Macías et al., Front. Plant Sci. 7 (1146): (2016)). AX produces novel iodine forms and therefore has a greater beneficial effect than KI or KIO₃. In addition, iodine compounds induce the expression of genes involved in the metabolism of a plant defense hormone and salicylic acid (SA). SA stimulates iodine uptake (Smoleń et al., Sci. Hortic. 188:89-96 (2015); Halka et al., Plant Physiol. Biochem. 144:35-48 (2019); Kiferle et al., Front. Plant Sci. 12 (616868): (2021)). The foregoing are incorporated by reference in their entirety.

In some embodiments, the invention provides hydrogen peroxide producing (HPP) enzymes. In certain embodiments, the HPP enzymes are oxidases that may be of the EX 1.1.3 subgenus. In particular embodiments, the oxidase may be EC 1.1.3.3 (malate oxidase), EC 1.1.3.4 (glucose oxidase), EC 1.1.3.5 (hexose oxidase), EC 1.1.3.6 (cholesterol oxidase), EC 1.1.3.7 (aryl-alcohol oxidase), EC 1.1.3.8 (L-gulonolactone oxidase), EC 1.1.3.9 (galactose oxidase), EC 1.1.3.10 (pyranose oxidase), EC 1.1.3.11 (L-sorbose oxidase), EC 1.1.3.12 (pyridoxine 4-oxidase), EC 1.1.3.13 (alcohol oxidase), EC 1.1.3.14 (catechol oxidase), EC 1.1.3.15 (2-hydroxy acid oxidase), EC 1.1.3.16 (ecdysone oxidase), EC 1.1.3.17 (choline oxidase), EC 1.1.3.18 (secondary-alcohol oxidase), EC 1.1.3.19 (4-hydroxymandelate oxidase), EC 1.1.3.20 (long-chain alcohol oxidase), EC 1.1.3.21 (glycerol-3-phosphate oxidase), EC 1.1.3.22, EC 1.1.3.23 (thiamine oxidase), EC 1.1.3.24 (L-galactonolactone oxidase), EC 1.1.3.25, EC 1.1.3.26, EC 1.1.3.27 (hydroxyphytanate oxidase), EC 1.1.3.28 (nucleoside oxidase), EC 1.1.3.29 (Nacylhexosamine oxidase), EC 1.1.3.30 (polyvinyl alcohol oxidase), EC 1.1.3.31, EC 1.1.3.32, EC 1.1.3.33, EC 1.1.3.34, EC 1.1.3.35, EC 1.1.3.36, EC 1.1.3.37 D-arabinono-1,4-lactone oxidase), EC 1.1.3.38 (vanillyl alcohol oxidase), EC 1.1.3.39 (nucleoside oxidase, H₂O₂ forming), EC 1.1.3.40 (D-mannitol oxidase), or EC 1.1.3.41 (xylitol oxidase).

The invention provides Free Radical Producing (FRP) enzymes in one of the sequential components of the solid fungicidal compositions. In some embodiments, the FRP is a peroxidase. Peroxidases are widely found in biological systems and form a subset of oxidoreductases that reduce hydrogen peroxide (H₂O2) to water in order to oxidize a large variety of aromatic compounds ranging from phenol to aromatic amines.

Peroxidases belong to the sub-genus EC 1.11.1. In certain embodiments, the EC 1.11.1 enzyme is The EC 1.11.1 enzyme can be more specifically, for example, EC 1.11.1.1 (NADH peroxidase), EC 1.11.1.2 (NADPH peroxidase), EC 1.11.1.3 (fatty acid peroxidase), EC 1.11.1.4, EC 1.11.1.5 (cytochrome-c peroxidase), EC 1.11.1.6 (catalase), EC 1.11.1.7 (peroxidase), EC 1.11.1.8 (iodide peroxidase), EC 1.11.1.9 (glutathione peroxidase), EC 1.11.1.10 (chloride peroxidase), EC 1.11.1.11 (L-ascorbate peroxidase), EC 1.11.1.12 (phospholipid-hydroperoxide glutathione peroxidase), EC 1.11.1.13 (manganese peroxidase), EC 1.11.1.14 (diarylpropane peroxidase), or EC 1.11.1.15 (peroxiredoxin).

In other embodiments, the peroxidase may also be further specified by function, e.g., a lignin peroxidase, manganese peroxidase, or versatile peroxidase. The peroxidase may also be specified as a fungal, microbial, animal, or plant peroxidase. The peroxidase may also be specified as a class I, class II, or class III peroxidase. The peroxidase may also be specified as a myeloperoxidase (MPO), eosinophil peroxidase (EPO), lactoperoxidase (LPO), thyroid peroxidase (TPO), prostaglandin H synthase (PGHS), glutathione peroxidase, haloperoxidase, catalase, cytochrome c peroxidase, horseradish peroxidase, peanut peroxidase, soybean peroxidase, turnip peroxidase, tobacco peroxidase, tomato peroxidase, barley peroxidase, or peroxidasin. In these particular embodiments, the peroxidase is a lactoperoxidase.

The lactoperoxidase/glucose oxidase (LP/GOX) antimicrobial system occurs naturally in bodily fluids such as milk, saliva, tears, and mucous (Bosch et al., J. Applied Microbiol., 89 (2), 215-24 (2000)). This system utilizes thiocyanate (SCN-) and iodide (I-), two naturally occurring compounds that are harmless to mammals and higher organisms (Welk et al. Archives of Oral Biology, 2587 (2011)). LP catalyzes the oxidation of thiocyanate and iodide ions into hypothiocyanite (OSCN-) and hypoiodite (OI-), respectively, in the presence of hydrogen peroxide (H₂O₂). The H₂O₂ in this system is provided by the activity of GOX on β-D-glucose in the presence of oxygen. These free radical compounds, in turn, oxidize sulfhydryl groups in the cell membranes of microbes (Purdy, Tenovuo et al. Infection and Immunity, 39 (3), 1187 (1983); Bosch et al., J. Applied Microbiol., 89 (2), 215-24 (2000), leading to impairment of membrane permeability (Wan, Wang et al. Biochemistry Journal, 362, 355-362 (2001)) and ultimately microbial cell death. Concentrations as low as 20 μM of hypothiocyanite and hypoiodite can result in inhibition of cell growth (Bosch, van Doorne et al. 2000). The LP/GOX system is effective on thiocyanate on its own; when paired with iodide, there is a synergistic effect that enhances biostatic and biocidal activity and extends the susceptible target range including Gram negative bacteria (e.g., E. coli, P. aerugenosa), Gram positive bacteria (e.g., S. aureus, Streptococcus spp.), and fungus (e.g., C. albicans) (Reiter, Marshall et al. Infection and Immunity, 13 (3), 800-807 (1976); Bosch et al., J. Applied Microbiol., 89 (2), 215-24 (2000); Welk et al. Archives of Oral Biology, 2587 (2011).) Furthermore, the LP/GOX system functions in two phases: (1) the generation and action of hypothiocyanite and hypoiodite on cell membranes, and then, when these compounds are depleted, (2) excess H₂O₂ builds up, enacting its own oxidative damage on cellular structures (Reiter, Marshall et al. 1976). The forgoing references are incorporated herein by reference in their entirety.

The enzyme system has been deployed and approved in the industry for biofilm control such as toothpaste and milk anti-spoiling agents. The system is largely non-specific and robust with few reaction requirements. One study found persistent biostatic and biocidal activity against Gram (−) and (+) bacteria and C. albicans after 18 months of re-inoculation every two months Bosch et al., J. Applied Microbiol., 89 (2), 215-24 (2000). The effective pH range is 3-7 with a peak LP activity at pH 5 (Reiter, Marshall et al. 1976; Purdy, Tenovuo et al. 1983). Higher activity is typically witnessed against bacteria at pH 3, but this is likely due to inhibition of growth by low pH (Reiter, Marshall et al. 1976). Other than pH, the only strict requirement for activity of the LP/GOX system is the presence of oxygen, without which GOX can't generate H₂O₂ from glucose. The forgoing references are incorporated herein by reference in their entirety.

LP/GOX has been described as a pesticide for microorganisms that include bacteria and fungi. (See U.S. Pat. No. 6,447,811, incorporated by reference herein in its entirety). Thus, in some embodiments, the invention described herein provides magnetically-immobilized pesticides in solid or liquid formulations. In some embodiments, the pesticides comprise a peroxidase enzyme that produces a free radical. In some embodiments, the peroxidase enzyme is lactoperoxidase. The pesticides further comprise a peroxide source that may include an enzyme that oxidizes glucose.

In some embodiments of the invention, the methods and compositions further comprise chemical pesticides such as fungicides or antibiotics.

In some embodiments of the invention, the chemical fungicide may be one or more of the following: mefenoxam, myclobutanil, chlorothalonil, prothioconazole, trifloxystrobin, propiconazole, mancozeb, Copper, methyl benzimidazole carbamates, dicarboximides, demethylation inhibitors (DMI), phenylamides (PA), amines, phosphorothiolates, dithiolanes, carboxamides, hydroxy-(2-amino-)pyrimidines, anilino-pyrimidines (AP), N-phenyl carbamates, quinone outside inhibitors (QOI), phenylpyrroles (PP), quinolines, aromatic hydrocarbons (AH), heteroaromatics, melanin biosynthesis inhibitors-dehydratase (MBI-D), hydroxyanilides, succinate biosynthesis inhibitors (SBI), polyoxins, phenylureas, quinone inside inhibitors (QiI), benzamides, enopyranuronic acid antibiotic, hexopyranosyl antibiotic, glucopyranosyl antibiotic, cyanoacetamide-oximes, carbamates, uncouplers of oxidative phosphorylation, organo tin compounds, carboxylic acids, heteroaromatics, phosphonates, phthalamic acids, benzotriazines, benzene-sulfonomides, pyridazinones, ATP production inhibitors, complex I of respiration inhibitors, carboxylix acid amides (CAA), tetracycline antibiotic, thiocarbamate, host plant defense inducers including salicylic acid pathway, fungicides with unknown target sites of action, fungicides with multi-site contact activity, mineral oils, organic oils, or potassium bicarbonate.

In some embodiments of the invention, the chemical antibiotic may be one or more of the following: chemical families of aminoglycosides, ansamycins, carbacephems, carbapenems, cephalosporins, glycopeptides, lincosamides, lipopeptides, macrolides, monolactams, nitrofurans, oxazolidinones, penicillins, polypeptide antibiotics, quinolones, fluoroquinolones, sulfonamides, tetracyclines. aminoglycosides, ansamycins, carbapenems, cephalosporins, glycopeptides, lincosamides, lipopeptides, macrolides, monobactams, nitrofurans, oxazolidinones, penicillins, polypeptides, quinolones, rifamycins, streptogramins, sulfonamides, tetracyclines, tuberactinomycins, or drugs with activity against mycobacteria. In preferred embodiments, the chemical antibiotic is ampicillin.

The invention provides that the biocidal compositions may be measured by its minimum inhibitory concentration (MIC) in the compositions and methods described herein. The MIC is the lowest concentration of a chemical that prevents visible growth of a bacterium, fungus, or oomycete. The MIC of the microbiocides may be determined, for instance, by preparing solutions of the chemical at increasing concentrations, incubating the solutions with the separate batches of cultured bacteria, and measuring the results using agar dilution or broth microdilution

The minimum bactericidal concentration (MBC) is the lowest concentration of an antibacterial agent required to kill a particular bacterium. It can be determined from broth dilution minimum inhibitory concentration (MIC) tests by subculturing on agar plates that do not contain the test agent. The MBC is identified by determining the lowest concentration of antibacterial agent that reduces the viability of the initial bacterial inoculum by ≥99.9%. The MBC is complementary to the MIC; whereas the MIC test demonstrates the lowest level of antimicrobial agent that inhibits growth, the MBC demonstrates the lowest level of antimicrobial agent that results in microbial death. This means that even if a particular MIC shows inhibition, plating the bacteria onto agar might still result in organism proliferation because the antimicrobial did not cause death. Antibacterial agents are usually regarded as bactericidal if the MBC is no more than four times the MIC. Microorganisms may survive microbiocides because they develop resistance to them.

The final chemical fungicide or antibiotic in the invention is at a final concentration of less than 1% or about 1%, 2%, 3%, 4%, 5%, 6%, 7%, 8%, 9% 10%, 20%, 30%, 40%, 50%, 60%, 70%, 80%, 90%, 100% of the minimum inhibitory concentration (MIC) or minimum bactericidal concentration (MBC).

The biocidal compositions of the invention can also be measured by their EC₅₀. The term half maximal effective concentration (EC₅₀) refers to the concentration of a drug, antibody or toxicant which induces a response halfway between the baseline and maximum after a specified exposure time. It is used herein as a measure of microbiocide potency. The EC₅₀ of a graded dose response curve therefore represents the concentration of a compound where 50% of its maximal effect is observed. The EC₅₀ of a quantal dose response curve represents the concentration of a compound where 50% of the population exhibit a response after a specified exposure duration. The microbiocides in the invention, including the fungicides and anbiotics, is at a final concentration of less than 1% or about 1%, 2%, 3%, 4%, 5%, 6%, 7%, 8%, 9% 10%, 20%, 30%, 40%, 50%, 60%, 70%, 80%, 90%, 100%. In other embodiments, the final chemical microbiocidal concentration is between about 100% and 500%, 500% and 1000%, 1000% and 2000%, 2000% and 2500%, 2500% and 5000%, 5000% and 10,000%.

The invention contemplates incorporating the biocidal compositions disclosed herein into multiple types of products. By way of example, the biocidal compositions may be comprised within one or more powders that are reconstituted with water to the working concentration. In additional embodiments, the powder comprises one or more enzyme concentrates or the enzymes are added prior to or at the time of use. Additionally, the invention contemplates an aqueous pre-concentrate that is diluted with water to the working concentration. In additional embodiments, the aqueous pre-concentrate comprises one or more enzyme concentrates or the enzymes are added prior to or at the time of use. As another example, the invention contemplates a liquid concentrate with the biocidal components, including enzymatic biocidal products, already produced and contained within the liquid concentrate. The concentrate is diluted with water prior to or at the time of use.

The invention provides inactive magnetically-immobilized enzymes. The enzymes may be inactive because they are not exposed to water, oxygen, substrates, or any combination thereof. In a preferred embodiment of the present invention, the magnetically-immobilized enzymes are in an oil base. This limits enzymatic activity prior to use. Activation of the immobilized enzymes occurs upon exposure to hydration and/or oxygen. In a more preferred embodiment, the magnetically-immobilized enzymes are in an oil base comprising an agent for emulsifying the oil in an aqueous solution to form an oil-in-water emulsion. In another more preferred embodiment, the oil is a mineral oil, vegetable oil, or animal oil. Exemplary mineral oils include paraffin oil and kerosene-type oils. Exemplary animal oils include fish oils such as herring and mackerel oil. Examples of vegetable oils are peanut oil, sesame oil, rape-seed oil, linseed oil, castor oil, soybean oil, corn germ oil, and cotton-seed oil.

In other embodiments, in order to further facilitate the distribution of the magnetically-immobilized enzymes over a surface, one or more spreading agents known in the art can further be added to the composition or the oil base. In some embodiments, the spreading agents are non-ionogenic surface tension-reducing substances. In preferred embodiments, the spreading agents are ethoxylated alcohols and phosphatidyl lipids.

In other embodiments, one or more adhesives can be added. Adhesives may help prevent the magnetically-immobilized enzymes from being rinsed off the plant by rain or other conditions. Adhesives are well known in the art. Examples are starch, gums such as xanthan gum, gum Arabic and carboxymethyl celluloses (CMCs).

The composition can be applied by means of coating, spraying, sprinkling, atomizing, overhead spraying, watering, immersing, overhead irrigation, and drip irrigation. A particularly advantageous method for applying the composition is spraying both by means of low volume methods (mist systems) and high volume methods. Drip irrigation can be used for culture systems on rockwool and other growth substrates. The enzyme systems according to the invention can also be used to disinfect drip irrigation systems. In both latter cases the presence of the oil base is not strictly necessary for an optimal activity. Immersion in a bath with the composition is particularly suitable for the treatment of plant parts, in particular harvestable parts, such as bulbs, tubers, fruits and the like.

Enzymes can be made commercially available in different forms. In a preferred embodiment, the peroxidase activity is delayed as long as possible because this increases the shelf-life of the product. The enzymatic activity starts upon exposure to both hydration (i.e. water) and oxygen. In the present case the glucose oxidase/glucose system is the hydrogen peroxide donor. In more preferred embodiments, the hydrogen peroxide donor is provided separately from the peroxidase. In addition, the oil base and the spreading agent can, if desired, also be packaged separately.

In another embodiment, a kit is provided comprising an optionally concentrated enzyme composition comprising a HPP enzyme (e.g. GOx plus glucose) and optionally a FRP enzyme (e.g. lactoperoxidase). In preferred embodiments, the kit may further comprise thiocyanate, iodide, oil, an emulsifier, or spreading agents. In more preferred embodiments, the ingredients are mixed with each other before use. In another embodiment, the kit may comprise one or more ingredients in a concentrated form for dilution or hydration prior to or concurrently with use.

In embodiments where β-D-Glucose is oxidized to H₂O₂, or where cellulose derived sugars are oxidized to H₂O₂, cellulase enzymes may be provided with the compositions of the invention. In some embodiments, the seed coating further comprises the cellulase.

In some embodiments, the cellulases are exocellulases, endocellulases, hemicellulases, or combinations thereof known in the art. Endocellulase (EC 3.2.1.4) randomly cleaves internal bonds at amorphous sites that create new chain ends. Exocellulase (EC 3.2.1.91) cleaves two to four units from the ends of the exposed chains produced by endocellulase, resulting in the tetrasaccharides or disaccharides, such as cellobiose. There are two main types of exocellulases [or cellobiohydrolases (CBH)]-CBHI works processively from the reducing end, and CBHII works processively from the nonreducing end of cellulose. Cellobiase (EC 3.2.1.21) or beta-glucosidase hydrolyses the exocellulase product into individual monosaccharides. Oxidative cellulases depolymerize cellulose by radical reactions, as for instance cellobiose dehydrogenase (acceptor). Cellulose phosphorylases depolymerize cellulose using phosphates instead of water.

In other embodiments, endocellulases may include EC 3.2.1.4, endo-1,4-beta-D-glucanase, beta-1,4-glucanase, beta-1,4-endoglucan hydrolase, celluase A, cellulosin AP, endoglucanase D, alkali cellulase, cellulase A 3, celludextrinase, 9.5 cellulase, avicelase, pancellase SS, and 1,4-(1,3, 1,4)-beta-D-glucan 4-glucanohydrolase). Cellulases enzymes are typically produced by fungi, bacteria, and protozoans of cellulose). Other names for ‘endoglucanases’ are: endo-1,4-beta-glucanase, carboxymethyl cellulase (CMCase), endo-1,4-beta-D-glucanase, beta-1,4-glucanase, beta-1,4-endoglucan hydrolase, and celludextrinase.

In some embodiments, the methods described herein use recombinant cells that express the enzymes used in the invention. Recombinant DNA technology is known in the art. In some embodiments, cells are transformed with expression vectors such as plasmids that express the enzymes. In other embodiments, the vectors have one or more genetic signals, e.g., for transcriptional initiation, transcriptional termination, translational initiation and translational termination. Here, nucleic acids encoding the enzymes may be cloned in a vector so that it is expressed when properly transformed into a suitable host organism. Suitable host cells may be derived from bacteria, fungi, plants, or animals as is well-known in the art.

In some embodiments, the invention provides that the matrix material is a biopolymer. Examples include the polysaccharides (e.g., cellulose, hemicellulose, xylan, chitosan, inulin, dextran, agarose, and alginic acid), polylactic acid, and polyglycolic acid. In other embodiments, the matrix material is a water-soluble cellulose derivative, a water-solvatable cellulose derivative, an alginate derivative, and a chitosan derivative.

The invention contemplates that the chemoenzymatic compositions are comprised within water-solvatable matrices. Non-limiting examples of such matrices include:
Polyacrylamides & Polymethacrylamides:
Poly(acrylamide)
Poly(N-isopropylacrylamide) (PNiPAM)
N-(2-Hydroxypropyl) methacrylamide (HPMA)
Polyacrylic Acid (PAA) & Poly-methacrylic Acid (PMAA):
Poly (acrylic acid)-block-poly(acrylamide)
Poly(N,N-dimethylaminoethyl methacrylate)
Amide Polymers:
Poly(N-vinyl acetamide)
Poly(acrylic acid)-block-poly(acrylamide)
Amine functional Polymers:
Poly(allylamine)
Poly(vinylamine) hydrochloride
Polyethylene Glycols (PEGs) & Derivatives:
Poly(ethylene oxide), =Poly(ethylene glycol)
Polyethyleneimines (PEI)
Polyvinyl alcohols (PVA)
Polyvinyl pyrrolidones (PVP):
Poly(N-vinylpyrrolidone)
Povidones
Poly(N-vinylpyrrolidone/vinyl acetate)
Polyvinyl amines
Polyphosphazenes
Polyethyl oxazolines (Polyoxazolines)
Poly(2-ethyl-2-oxazoline)
Polyvinyl acetates
Poly-ethylene-vinyl acetate and Poly-ethylene-vinyl alcohols
Poly(vinyl acetate)
Styrenic Polymers
Poly(styrene sulfonic acid), sodium salt or free acid
Miscellaneous Polymers:
Poly(maleic acid)
Poly(vinyl phosphoric acid), sodium salt and its derivatives
Poly(vinylsulfonic acid), sodium salt
Poly(vinylphosphonic acid)
Poly(2-vinylpyridine)
Poly(2-hydroxyethyl methacrylate/methacrylic acid)
Poly(2-vinyl-1-methylpyridinium bromide)
Poly(2-vinylpyridine N-oxide)
Poly(4-vinylpyridine)
Poly(ethylene oxide-b-propylene oxide)
Divinyl Ether-Maleic Anhydride (DIVEMA)
Poly (glycerol adipate-co-o-pentadecalactone)
Poly (1,3-propanediol adipate-co-o-pentadecalactone)
Poly (lactide-co-glycolide)
Poly (lactic-co-glycolic) acid
Poly (lactic acid)
Shellac
Plant hydrocolloids
Polyvinyl methyl ethers
Carboxy-vinyl polymers
Detergents
Poly(oxyethylene) sorbitan monolaureate (Tween 20)
Carbohydrates (Polysaccharides):
Dextran
Dextran polymer
Carboxymethylcellulose (CMC), sodium salt
Hydroxypropyl cellulose (HPC)
Hydroxypropyl methyl cellulose (HPMC)
Methyl cellulose (MC)
Cellulose, hydroxyethyl ether
Hyaluronic acid (HA)
Cellulose ethers
Alginates
Xanthan gum
Pectins
Chitosan derivatives
Carrageenan
Guar Gum
Starch or Starch based derivatives
Gum Arabic
Proteins and peptides:
Albumin
Gelatin
B-Lactoglobulin
Lactoferrin
Poly(L-lysine hydrobromide)
Poly(L-glutamic acid)
Inorganic water-soluble polymers:
Bentonite
Aluminum magnesium silicate
Laponite
Hectorite
Silicic anhydride
Polyphosphates

### DNA or RNA

In some embodiments, the matrix comprises cellulose. Cellulose is an organic compound with the formula (C₆H₁₀O₅)n, a polysaccharide consisting of a linear chain of several hundred to many thousands of β(1→4) linked D-glucose units. The cellulose used in the invention may be obtained or derived from plant, algal, or microbial sources. In some embodiments, the invention provides cellulose derivatives known in the art. The hydroxyl groups (—OH) of cellulose can be partially or fully reacted with reagents known in the art. In preferred embodiments, the cellulose derivatives are cellulose esters and cellulose ethers (—OR). In more preferred embodiments, the cellulose derivatives are cellulose acetate, cellulose triacetate, cellulose proprionate, cellulose acetate proprionate (CAP), cellulose acetate butyrate (CAB), nitrocellulose (cellulose nitrate), cellulose sulfate, methylcellulose, ethylcellulose, ethyl methyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose, and carboxymethyl cellulose (CMC).

In some embodiments, the matrix comprises carboxymethyl cellulose. Carboxymethyl cellulose (CMC) or cellulose gum [1] is a cellulose derivative with carboxymethyl groups (—CH2-COOH)) bound to some of the hydroxyl groups of the glucopyranose monomers that make up the cellulose backbone. It is synthesized using techniques known in the art, e.g., by the alkali-catalyzed reaction of cellulose with chloroacetic acid. The polar (organic acid) carboxyl groups render the cellulose soluble and chemically reactive. The functional properties of CMC depend on the degree of substitution of the cellulose structure (i.e., how many of the hydroxyl groups have taken part in the substitution reaction), as well as the chain length of the cellulose backbone structure and the degree of clustering of the carboxymethyl substituents.

In some embodiments, the matrix comprises hydroxypropyl cellulose (HPC). HPC is a derivative of cellulose with both water solubility and organic solubility. HPC is an ether of cellulose in which some of the hydroxyl groups in the repeating glucose units have been hydroxypropylated forming —OCH2CH(OH)CH3 groups using propylene oxide. The average number of substituted hydroxyl groups per glucose unit is referred to as the degree of substitution (DS). Complete substitution would provide a DS of 3. Because the hydroxypropyl group added contains a hydroxyl group, this can also be etherified during preparation of HPC. When this occurs, the number of moles of hydroxypropyl groups per glucose ring, moles of substitution (MS), can be higher than 3. Because cellulose is very crystalline, HPC must have an MS about 4 in order to reach a good solubility in water. HPC has a combination of hydrophobic and hydrophilic groups, so it has a lower critical solution temperature (LCST) at 45° C. At temperatures below the LCST, HPC is readily soluble in water; above the LCST, HPC is not soluble. HPC forms liquid crystals and many mesophases according to its concentration in water. Such mesophases include isotropic, anisotropic, nematic and cholesteric. The last one gives many colors such as violet, green and red.

In some embodiments, the matrix comprises methyl cellulose. Methyl cellulose (or methylcellulose) is derived from cellulose. It is a hydrophilic white powder in pure form and dissolves in cold (but not in hot) water, forming a clear viscous solution or gel. Methyl cellulose does not occur naturally and is synthetically produced by heating cellulose with caustic solution (e.g. a solution of sodium hydroxide) and treating it with methyl chloride. In the substitution reaction that follows, the hydroxyl residues (—OH functional groups) are replaced by methoxide (—OCH3 groups).

Different kinds of methyl cellulose can be prepared depending on the number of hydroxyl groups substituted. Cellulose is a polymer consisting of numerous linked glucose molecules, each of which exposes three hydroxyl groups. The Degree of Substitution (DS) of a given form of methyl cellulose is defined as the average number of substituted hydroxyl groups per glucose. The theoretical maximum is thus a DS of 3.0, however more typical values are 1.3-2.6.

In some embodiments, the matrix comprises alginate. Alginate, also called Alginic acid, and algin, is an anionic polysaccharide distributed widely in the cell walls of brown algae. When bound with water it forms a viscous gum. In extracted form it absorbs water quickly; it is capable of absorbing 200-300 times its own weight in water. It is sold in filamentous, granular or powdered forms. The invention provides matrix materials of known alginate and alginate-derived materials. In preferred embodiments, the alginate-derived materials include alginate-polylysine-alginate (APA), Alginate/Poly-l-lysine/Pectin/Poly-1-lysine/Alginate (APPPA), Alginate/Poly-1-lysine/Pectin/Poly-l-lysine/Pectin (APPPP), and Alginate/Poly-L-lysine/Chitosan/Poly-l-lysine/Alginate (APCPA), alginate-polymethylene-co-guanidine-alginate (A-PMCG-A), hydroxymethylacrylate-methyl methacrylate (HEMA-MMA), multilayered HEMA-MMA-MAA, polyacrylonitrile-vinylchloride (PAN-PVC).

In some embodiments, the matrix comprises chitosan. Chitosan is a linear polysaccharide composed of randomly distributed β-(1-4)-linked D-glucosamine (deacetylated unit) and N-acetyl-D-glucosamine (acetylated unit). The amino group in chitosan has a pKa value of ˜6.5, which leads to a protonation in acidic to neutral solution with a charge density dependent on pH and the % DA-value. This makes chitosan water soluble and a bioadhesive which readily binds to negatively charged surfaces such as mucosal membranes. It is produced commercially by deacetylating chitin, which is the structural element in the exoskeleton of crustaceans (such as crabs and shrimp) and cell walls of fungi, with sodium hydroxide. Chitosan is used in agriculture as a seed treatment and biopesticide. In winemaking, it is used as a fining agent, also helping to prevent spoilage. It is also used in bandages to reduce bleeding and as an antibacterial agent. It is also be used to help deliver drugs through the skin.

In other embodiments, the matrix materials may be acrylonitrile/sodium methallylsuflonate, (AN-69), polyethylene glycol/poly pentamethylcyclopentasiloxane/polydimethylsiloxane (PEG/PD5/PDMS), poly JVjiV-dimethyl acrylamide (PDMAAm), siliceous encapsulates, and cellulose sulphate/sodium alginate/polymethylene-co-guanidine (CS/A/PMCG).

In some embodiments, the invention provides antimicrobial compositions that are used, inter alia, to treat plants, fruits, or seeds. Any plants, fruits, or seeds that are vulnerable to pathogens that respond to the enzyme systems disclosed herein would benefit. In some embodiments, the plants may be for vegetables, fruits, field crops, and flowers. In other embodiments, the invention provides antimicrobial compositions that are used, inter alia, for bedding for industrially or commercially relevant domesticated animals and products derived therefrom. Many domesticated animals are known in the art. In other embodiments, the invention provides fungicidal compositions that are used, inter alia, for wound dressings. Many wound dressings are known in the art. The invention provides fabrics that resist pathogens or contaminants that respond to the enzyme systems disclosed herein. The fabrics comprise the fungicidal compositions described herein.

In order that the invention described herein may be more fully understood, the following examples are set forth. It should be understood that these examples are for illustrative purposes only and are not to be construed as limiting this invention in any manner.

### Example 1: Reagents and Materials

The following chemicals reagents are used to synthesize the biocidal agents: Potassium Iodide (KI, Deepwater Chemicals, 101.24, CAS No. 7681-11-0), Potassium Thiocyanate (KSCN, Fisher Scientific, AC196585000, CAS No. 333-20-0), Glucose (D-Glucose, 99%, anhydrous, Acros Organics, 10032070, CAS No. 50-99-7), Sodium Acetate Trihydrate (Allan Chemical Corp., AAA1623036 CAS No. 6131-90-4), pressurized oxygen (Airgas OX 200), and hematite scaffold (average particle size, 40 μm, Powdertech, Rhosmedre, Wrexham, United Kingdome). All water is obtained from an Express Water RO System water purifier (Express Water, RODI10-D).

Bovine milk enzyme Lactoperoxidase (LP, E.C. 1.11.1.7) is purchased from Tatua Dairy Company (Morrinsville, New Zealand). Fungal enzyme Glucose Oxidase (GOx, β-D-Glucose: Oxygen 1-oxidoreductase, Source: Aspergillus niger, E.C. 1.1.3.4) is purchased from Millipore Sigma.

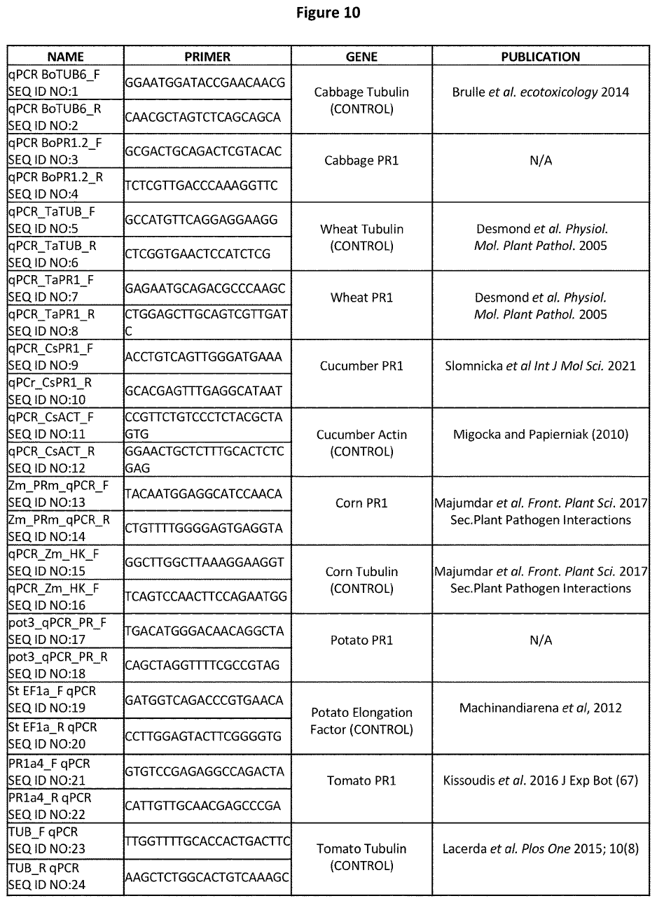

For qPCR analysis the following materials and reagents were used: NEB Monarch Total RNA Miniprep Kit (T2010S), NEB Luna Universal One-Step RT-qPCR Kit (E3005S), qPCR primers (SEQ ID NOS: 1-24) from FIG. 10.

### Example 2 Formulations

Formulation A: AX Biocidal Production from Concentrate.

An AX biocidal formulation (35 mM KI, 8 mM KSCN, 300 mM Glucose, and 200 mM Sodium Acetate, 20 U/mL Glucose Oxidase, and 25 U/mL Lactoperoxidase) is produced from a 10× salt-sugar concentrate. It is diluted with water to the working concentration and subsequent addition of the two enzyme solutions.

The 10× concentrate is composed of 3.0 M Glucose, 2.0 M Sodium Acetate, 350 mM Potassium Iodide, and 80 mM Potassium Thiocyanate. The concentrate is prepared by the dissolution of 54 g of D-Glucose and 27.2 g of Sodium Acetate Trihydrate in 100 mL of deionized water. The solution is heated to 70° C. with vigorous shaking to fully dissolve the solids. 5.8 g of Potassium Iodide and 777 mg of Potassium Thiocyanate is then added to the solution and allowed to dissolve overnight on a rotator.

A 33× (5.0 mg/ml, 820 U/ml) solution of LP (Lactoperoxidase, from bovine milk, activity 164 U/mg) is prepared in 200 mM Acetate Buffer at pH 5.5 by addition of 30.5 mL of 200 mM Acetate Buffer (pH 5.5) to 152 mg of LP. The LP solution is stored in a 4° C. refrigerator until use.

A 74× (10 mg/ml, 1480 U/ml) solution of GOx (Glucose Oxidase, from Aspergillus niger, activity 148 U/mg) is prepared by addition of 13.5 mL of Deionized water to 135 mg of GOx. The GOx solution is stored in a 4° C. refrigerator until use.

The AX biocidal formulation is produced by adding 100 mL of the 10× concentrate to 856 mL of Deionized water in a 2 L media bottle equipped with a magnetic stir bar. The mixture is stirred until homogeneous. 30.5 mL of the 33× LP solution is added to the stirring 1× solution. Finally, 13.5 mL of the 74× GOx solution is added to the solution. The bottle is closed firmly, and the reaction mixture is stirred for 24 hours at 25° C. using a magnetic stirrer.

### Formulation B: Enzyme-Free Biocide Produced Using In-Flow Biocatalytic Reactor

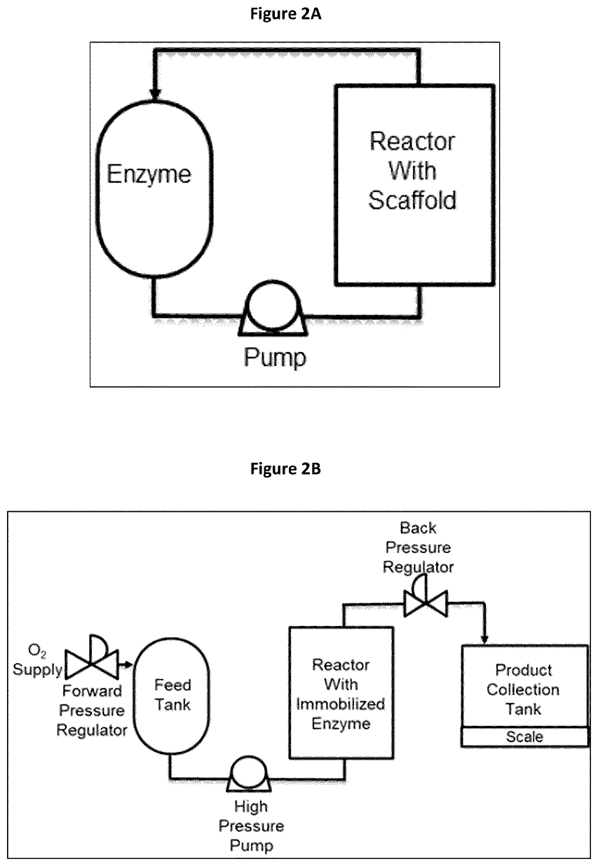

An enzyme-free biocidal product is made using immobilized enzymes that release their products into the biocidal solution. Lactoperoxidase (50 mg) and Glucose oxidase (50 mg) dissolved in 10 ml of water are recirculated with a peristaltic pump through a 10 ml packed bed reactor filled with hematite scaffold (average particle size, 40 μm, Powdertech, Rhosmedre, Wrexham, United Kingdome) at ten bed volumes per hour until enzyme immobilization has reached >98% as established by the Bradford assay. The process flow diagram for enzyme immobilization is shown in FIG. 2A.

After immobilization, the feed solution (35 mM KI, 8 mM KSCN, 300 mM glucose, and 200 mM acetate buffer at a pH of 5.5) is pumped with a high-pressure pump through the column for the continuous generation of the biocidal solution. The feed tank is pressurized with oxygen to 800 psi to increase the oxygen concentration in the feed. The feed solution is pumped at a flow rate of two bed volumes per hour. Immobilized glucose oxidase converts glucose and oxygen to Hydrogen peroxide and lactoperoxidase converts KI and KSCN to OI⁻ and OSCN⁻. These further react to form a mixture of oxidative iodo species including I₂, I₃⁻, I₂SCN, I(SCN)₂⁻. (See, e.g., FIG. 1B.) The biocidal solution that includes OI- and OSCN- products is collected (FIG. 2B). The biocidal solution may be further formulated into products for agricultural applications.

### Formulation C: Production of AX Biocidal Agents

An AX Biocidal formulation (35 mM KI, 8 mM KSCN, 300 mM Glucose, and 200 mM Sodium Acetate, 20 U/mL Glucose Oxidase, and 25 U/mL Lactoperoxidase) was produced from a 1× salt-sugar solution and subsequent addition of the two enzyme solutions.

A 33× (5.0 mg/ml, 820 U/ml) solution of LP (Lactoperoxidase, from bovine milk, activity 164 U/mg) was prepared in 200 mM Acetate Buffer at pH 5.5 by addition of 30.5 mL of 200 mM Acetate Buffer (pH 5.5) to 152 mg of LP. The LP solution was stored in a 4° C. refrigerator until use.

A 74× (10 mg/ml, 1480 U/ml) solution of GOx (Glucose Oxidase, from Aspergillus niger, activity 148 U/mg) was prepared by addition of 13.5 mL of Deionized water to 135 mg of GOx. The GOx solution was stored in a 4° C. refrigerator until use.

The AX biocidal formulation was produced by addition of 5.8 g of Potassium Iodide, 777 mg of Potassium Thiocyanate, 54 g of D-Glucose, and 26.4 g of Sodium Acetate Trihydrate to a 2 L media bottle equipped with a magnetic stir bar. To this, 856 mL of DI water was added, and the mixture was stirred until homogeneous. The pH was measured to be 8.2. Next, 30.5 mL of the 33× LP solution was added to the stirring solution followed by addition of 13.5 mL of the 74× GOx solution. The bottle was closed firmly, and the reaction mixture was stirred for 24 hours at 25° C. using a magnetic stirrer. After 24 hours, the solution reached a pH of 5.5.

Formulation D: AX Biocidal Agent with Enzymes and Water-Solvatable Matrix

An AX Biocidal formulation was produced from a 1× salt-sugar solution and the subsequent addition of the two enzyme solutions. It comprises 35 mM KI, 8 mM KSCN, 300 mM Glucose, 200 mM Sodium Acetate, 20 U/mL Glucose Oxidase formulated in carboxymethylcellulose, and 25 U/mL Lactoperoxidase formulated in carboxymethylcellulose.

A 33× (5.0 mg/ml, 820 U/ml) solution of LP formulated in carboxymethylcellulose is prepared in 200 mM Acetate Buffer at pH 5.5 by addition of 30.5 mL of 200 mM Acetate Buffer (pH 5.5) to 152 mg of LP. The LP solution is stored in a 4° C. refrigerator until use.

A 74× (10 mg/ml, 1480 U/ml) solution of GOx formulated in carboxymethylcellulose is prepared by addition of 13.5 mL of Deionized water to 135 mg of GOx. The GOx solution is stored at 4° C. until use.

The AX biocidal formulation is produced by addition of 5.8 g of Potassium Iodide, 777 mg of Potassium Thiocyanate, 54 g of D-Glucose, and 26.4 g of Sodium Acetate Trihydrate to a 2 L media bottle equipped with a magnetic stir bar. To this, 856 mL of DI water is added and the mixture is stirred until homogeneous. The pH is adjusted to 8.2. Next, 30.5 mL of the 33× LP solution is added to the stirring solution followed by addition of 13.5 mL of the 74× GOx solution. The bottle is closed firmly, and the reaction mixture is stirred for 24 hours at 25° C. using a magnetic stirrer. After 24 hours, the solution is adjusted to a pH of 5.5.

### Example 3: Measuring Systemic Acquired Response (SAR) Gene Activation

Two methods of application were used for AX formulations: (1) AX was applied at a rate of 50 gpa (gallons per acre) with a hand-held pressurized CO₂ sprayer at 37 psi, or (2) 10 ml of AX was applied to each plant with a hand sprayer. All AX formulations were made using the production method described in Formulation C. RNA was extracted from leaf tissue samples using the NEB Monarch Total RNA Miniprep Kit and qPCR performed from the RNA with NEB Luna Universal One-Step RT-qPCR kit run with QuantStudio 7 Pro from ThermoFisher. A housekeeping gene for each species, either Actin (ACT), Tubulin (TUB) or Elongation Factor 1 (EF1), was used as a reference gene. It was compared with a species-specific SAR gene, Pathogenesis-Related 1 (PR1).

The ΔΔCt method was performed for analysis. The formula utilizes expression values from control samples and genes to calculate relative fold gene expression by quantitative PCR (qPCR). The ΔΔCt method (2-ΔΔCt) is a well-known and accepted method for quantifying gene expression. (Livak K J, Schmittgen T D., Methods 25 (4): 402-8 (2001). doi: 10.1006/meth.2001.1262. PMID: 11846609, incorporated herein by reference in its entirety.) The gene-specific primers are listed in FIG. 10.

### Example 4: SAR-Based Plant Survival

Application of AX with and without LP induced SAR activation and plant immune responses. All AX formulations were made using the protocol described Formulation C. The AX formulations were applied to plants and SAR gene expression was measured as described in Example 3. Primers for qPCR marker gene analysis are listed in FIG. 10.

### Example 5: Inducing Systemic Acquired Response (SAR) Gene Activation

Tomato. AX Biocidal formulations surprisingly induced the SAR marker gene pathogenesis related-1 (PR1) in tomato plants. The formulations were as follows:

### Formulations

70 mM KI, 16 mM KSCN; 40 U/ml Gox 50 U/ml LP
70 mM KI, 16 mM KSCN; 40 U/ml Gox

The formulations were optimized for SAR gene activation and pathogen inhibition. It was determined that a salt formulation comprising about 70 mM KI and 16 mM KSCN resulted in optimal biocidal efficacy. Using in vitro plate-based protocols of in-door tomato efficacy trials with 40 U/ml GOx and 50 U/ml LP, it was observed that if the salt concentration is reduced by 2-fold to 35 mM KI and 8 mM KSCN, the enzyme concentrations should also be reduced by 2-fold to achieve optimal biocidal efficacy. The enzyme concentrations were therefore linearly adjusted with total salt concentration (KI+KSCN) and as a first best guess. It was further noted that the LP concentration may be reduced to avoid phytotoxicity without overly compromising the biocidal activity.

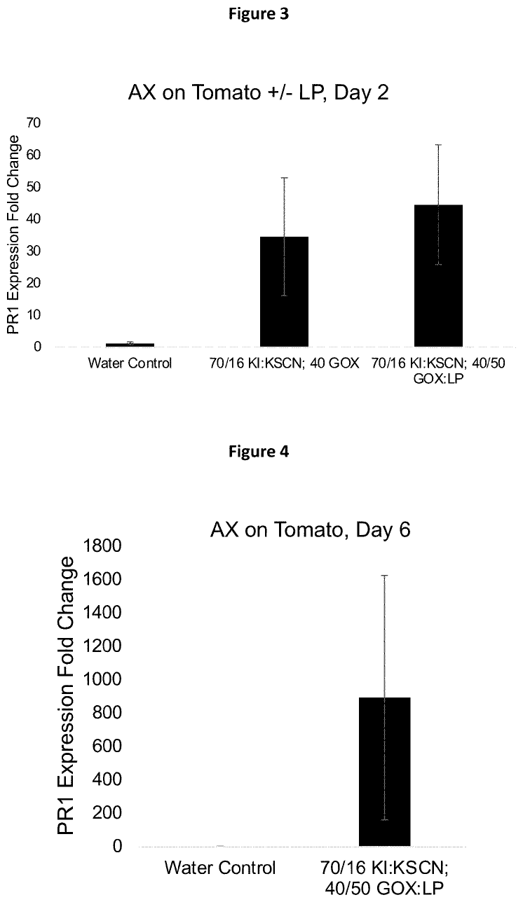

Two days post-application, samples were taken and analyzed for PRI gene expression using primers identified as SEQ ID NOS: 21 and 22. Leaves sprayed with water were used as controls. The 70/16 KI:KSCN 40 GOX induced an approximately 28-fold increase in PRI expression over the water control. The 70/16 KI:KSCN 40/50 GOX:LP induced an approximately 36-fold increase in PRI expression. (FIG. 3). Moreover, 70/16 KI:KSCN 40/50 GOX:LP induced an approximately 592-fold increase in PR1 expression six days post-application compared to the water control (FIG. 4).

Potato. AX biocidal formulation 70 mM KI, 16 mM KSCN; 40 U/ml Gox induced the PR1 in potato plants. It was experimentally determined that 40 U/ml of GOx is matched best with 70 mM KI and 16 mM KSCN. Without being bound by theory, this GOx concentration may produce hydrogen peroxide (H₂O2) at a rate that is optimal for it to either further react chemically (non-enzymatically) with KI or enzymatically via LP. The LP concentration of 50 U/ml was initially chosen to be slightly higher than the turnover rate of GOx (40 U/ml). The LP concentration may, however, be reduced to achieve improved results.

Two days post-application, samples were taken and analyzed for PRI gene expression using primers identified as SEQ ID NOS: 17 and 18. Leaves sprayed with water were used as controls. The 70/16 KI:KSCN 40 GOX induced an approximately 226-fold increase in potato PRI expression over the water control. (FIG. 5).

Cabbage. AX biocidal formulations were optimized for SAR gene expression in cabbage plants. The following formulations were used:

### Formulations

40 U/ml Gox+50 U/ml LP with Li700 (0.1%) penetrant (phosphatidylcholine, methylacetic acid and alkyl polyoxyethylene ether—80%; Loveland Inc.)
35 mM KI 8 mM KSCN with Li700 (0.1%) penetrant (phosphatidylcholine, methylacetic acid and alkyl polyoxyethylene ether—80%; Loveland Inc.)
18 mM KI 4 mM KSCN; 40 U/ml Gox+50 U/ml LP with Li700 (0.1%) penetrant (phosphatidylcholine, methylacetic acid and alkyl polyoxyethylene ether—80%; Loveland Inc.)
35 mM KI 8 mM KSCN; 40 U/ml Gox+50 U/ml LP with Li700 (0.1%) penetrant (phosphatidylcholine, methylacetic acid and alkyl polyoxyethylene ether—80%; Loveland Inc.)

LI 700® is a commercial adjuvant (Loveland Inc, CA Reg. No. 34704-50035; WA Reg. No. 34704-04007, Loveland, Colorado). It is a soy-oil derived, non-ionic penetrating surfactant that reduces off-target spray drift and reduces spray water pH. The formulations were applied to cabbage plants and three days post application samples were taken and analyzed for PRI expression using the primers SEQ ID NOS: 3 and 4. Leaves were sprayed with water or Li700 for controls.

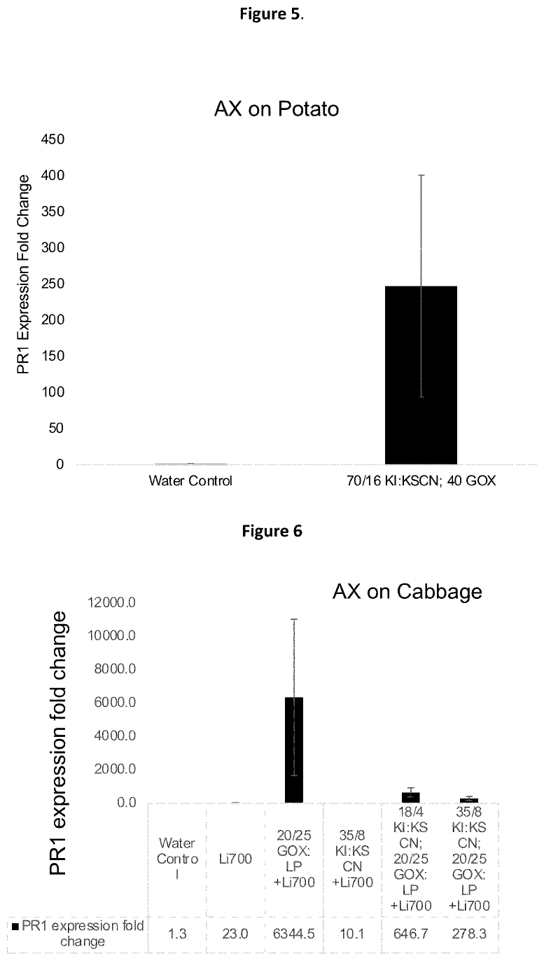

Surprisingly, the 40 U/ml Gox+50 U/ml LP with Li700 (0.1%) penetrant (20/25 GOX:LP plus Li700 in FIG. 6) showed an approximately 5,068-fold increase in PR1 Expression compared to the water control. The next runner up was the 18/4 KI:KSCN; 20/25 GOX:LP+Li700 with an approximately 516-fold increase compared to the water sprayed control. Third was the 35/8 KI:KSCN; 20/25 GOX:LP+Li700 with an approximately 222-fold increase compared to the water control. Thus, was shown that the ration of GOx to LP and KI to KSCN may be optimized to achieve a significantly-increased SAR response (FIG. 6).

Cucumber. AX biocidal formulation 35 mM KI, 8 mM KSCN; 40 U/ml Gox, and 50 U/ml LP induced the SAR marker PRI as measured in cucumber plants. This rate was chosen based on the plant phytotoxicity response, the salt and enzyme ratio showed minimal phytotoxicity. A formulation of 70 mM KI, 16 mM KSCN, 40 U/ml Gox, and 50 U/ml LP induced a high phytotoxicity and was therefore not used.

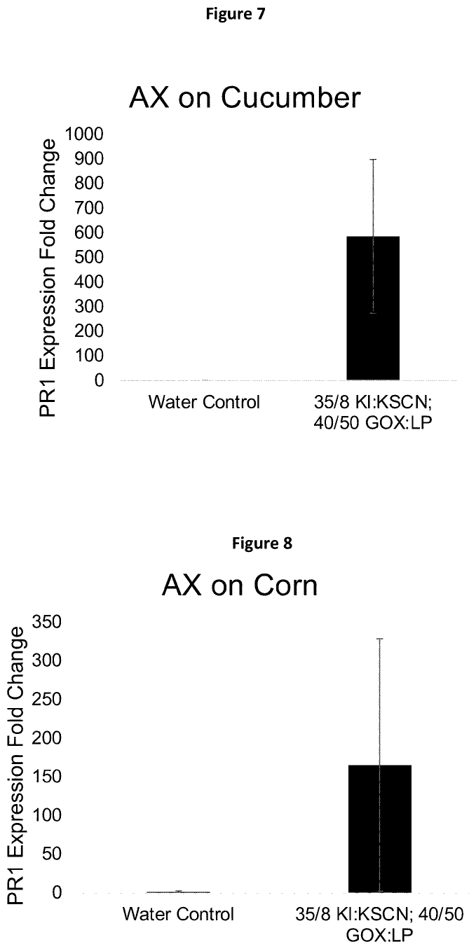

Six days post-application, samples were taken and analyzed for PRI gene expression using primers identified as SEQ ID NOS: 9 and 10. Leaves sprayed with water were used as controls. The 35/8 KI:KSCN 40/50 GOX:LP GOX induced an approximately 552-fold increase in PRI expression when compared to the water control. (FIG. 7).

Corn. AX biocidal formulation 35 mM KI 8 mM KSCN; 40 U/ml Gox 50 U/ml LP induced the SAR marker PRI was measured in corn plants. This rate was chosen based on the plant phytotoxicity response, the salt and enzyme ratio showed minimal phytotoxicity. A formulation of 70 mM KI, 16 mM KSCN, 40 U/ml Gox 50 U/ml, and LP induced a high phytotoxicity and was therefore not used.

Two days post-application, samples were taken and analyzed for PR1 gene expression using primers identified as SEQ ID NOS: 13 and 14. Leaves sprayed with water were used as controls. A 35/8 KI:KSCN; 40/50 GOX:LP GOX formulation induced an approximately 93-fold increase in PRI expression compared to the water control. (FIG. 8).

Wheat. AX biocidal formulation 35 mM KI 8 mM KSCN; 40 U/ml Gox 50 U/ml LP induced the SAR marker PRI was measured in wheat plants. This rate was chosen based on a plant phytotoxicity response; the salt and enzyme ratio showed minimal phytotoxicity.

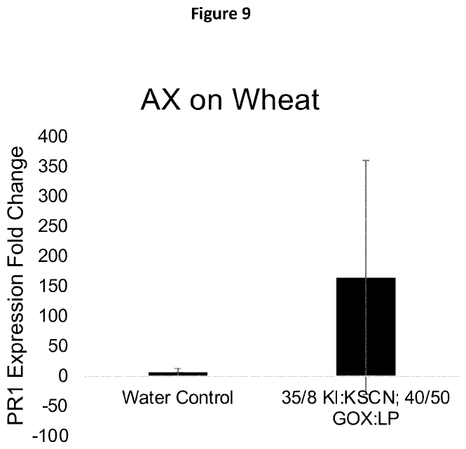

Four days post-application, samples were taken and analyzed for PRI gene expression using primers identified as SEQ ID NOS: 7 and 8. Leaves sprayed with water were used as controls. The 35/8 KI:KSCN 40/50 GOX:LP GOX induced an approximately 25-fold increase in PRI compared to the water control. (FIG. 9).

### Example 6: Disease Suppression in Field Cabbage

AX suppressed black rot caused by Xanthomonas campestris pv campestris in cabbage planted in fields. Cabbage cultivar ‘Bravo’ transplants were planted into a Palmyra gravel loam using a complete randomized block design with four replicates. Plants were fertilized with 19-19-19 nitrogen, phosphorous and potassium (NPK) respectively at a rate of 240 pounds (lbs) of fertilizer per acre, and plants were hand-watered as needed. Plots were 25 feet long and rows were 7 feet apart. Plots consisted of 10 plants, and all data were collected from the center 5 plants. Kocide 3000® (Certis LLC, MD, EPA Reg. No. 91411-11-70051) was applied as a commercial standard control using Silwet® (Helena Ag LLC, TN, CA Reg. No. 5905-50073-AA) at 0.1% as a wetting agent. A non-treated control was also included in the trial.

AX formulations were applied at a rate of 50 gallons per acre (gpa) to cabbage plots with a CO2 hand-held pressurized (37 psi) sprayer on Days 10, 15, 24, 31, 38 and 45 days post-planting. Adjuvants Li700 (0.1%) penetrant (phosphatidylcholine, methylacetic acid and alkyl polyoxyethylene ether—80%; Loveland Inc.) and Tactic (0.1%) spreader sticker (Synthetic latex, 1,2-propanediol, alcohol ethoxylate, silicone polyether copolymer-64%; Loveland Inc.) were applied with the AX formulations to increase penetration due to the hydrophobic nature of cannage leaf tissue. The spray boom consisted of 2 hollow cone nozzles (=TeeJet TXVS18) set on the 18 inch center.

### Formulations

35 mM KI 8 mM KSCN; 20 U/ml Gox+25 U/ml LP with Li700 (0.1%) penetrant (phosphatidylcholine, methylacetic acid and alkyl polyoxyethylene ether—80%; Loveland Inc.)
35 mM KI 8 mM KSCN; 20 U/ml Gox+Li700 (0.1%) penetrant (phosphatidylcholine, methylacetic acid and alkyl polyoxyethylene ether—80%; Loveland Inc.)
35 mM KI 8 mM KSCN; 20 U/ml Gox+Tactic (0.1%) spreader sticker (Synthetic latex, 1,2-propanediol, alcohol ethoxylate, silicone polyether copolymer—64%; Loveland Inc.)

This rate was chosen based on plant phytotoxicity response; the salt and enzyme ratio showed minimal phytotoxicity. A formulation of 70 mM KI, 16 mM KSCN; 40 U/ml Gox 50, U/ml LP induced high phytotoxicity and was therefore not used.

Cabbage plants were inoculated with Xanthomonas campestris pv campestris grown from cultures. Isolated bacteria was sprayed on cabbage plants using a handheld sprayer. Following inoculations, cabbages were watered to maintain moisture.

Plants were rated 23, 38, and 60 days post-planting for the percent leaf area with symptoms of black rot disease. On all rating dates, AX formulations and Kocide 3000 reduced the severity of Black rot compared to the nontreated control. The final rating) was 3 weeks after the final application. Cabbage foliage showed no phytotoxicity from AX formulations in this study. Thus, cabbage appears unaffected by these formulations. In this trial, the AX formulations prepared with Gox only were as effective as the Gox+LP. This indicated that LP has an additive effect but is not required for disease suppressive activity.

Data were analyzed with JMP-SAS for analysis of variance and significant differences, Tukey's HSD was used to assess multiple comparison of means (0.05) (FIG. 11).

### Example 7: Disease Suppression in Field Cucumber

AX formulations showed efficacy against downy mildew on cucumbers caused by Pseudoperonospora cubensiswas. Cucumber cultivar ‘Straight Eight’ seeds were planted into a Palmyra gravel loam using a complete randomized block design with four replicates. Plants were fertilized with 19-19-19 nitrogen, phosphorous and potassium (NPK) respectively at a rate of 240 pounds (lbs) of fertilizer per acre, and plants were hand-watered as needed. Plots were 20 feet long and rows were 7 feet apart. Plots consisted of 10 plants, and all data were collected from the center 5 plants. Stargus (ProFarm Group, CA) biological standard and a non-treated control were included in the trial. Plots were not inoculated. Infection was induced from natural field inoculum.

Products were applied using a CO₂ hand-held pressurized sprayer, that was calibrated to deliver 50 gallons per acre (gpa) at 42 psi. For the first application, the spray boom consisted of 2 hollow cone nozzles (=TeeJet TXVS18) set on the 18-inch center. Cucumber plants were tied to bamboo stakes that were approximately 24 inches tall. For subsequent application, a 3 nozzle boom was used. A single nozzle treated the top of the plants and 2 drop-down nozzles applied a spray to the plants within each plot. Applications were made beginning on Day 39 post-planting after the first lesions of downy mildew were visible and on Days 45 and 52 post-planting. All rates were lowered for the last application on Day 52 post-planting due to phytotoxicity. This indicated that the formulation is critical for effective disease control due to the risk for phytotoxicity.

### Formulations

70 mM KI 16 mM KSCN; 20 U/ml Gox 25 U/ml LP; 8/3 and 8/10; 35 mM KI 8 mM KSCN; 20 U/ml Gox 25 U/ml LP on 8/16
35 mM KI 8 mM KSCN; 20 U/ml Gox 25 U/ml LP; 8/3 and 8/10; 35 mM KI 8 mM KSCN; 10 U/ml 25 U/ml LP on 8/16

Plants were rated for the percent leaf area with symptoms of downy mildew. All ratings were from evaluating the upper leaf surface for the percent leaf area with symptoms of downy mildew. A single rating was recorded from each of 5 plants within the center of each plot.

The weather was extremely conducive for downy mildew. The disease developed rapidly on the cucumber foliage during this trial. Both AX formulations delayed the development of downy mildew as no symptoms were visible eight days after the first evaluation. At 12 days after the first application, downy mildew on the foliage was moderate within all treatments, however the severity of downy mildew within each AX treated plot was numerically reduced, compared to the nontreated plots. At 20 days after the first application, the severity of downy mildew on the foliage treated with each AX formulation was reduced significantly compared to both the non-treated and the Stargus® treated plants.

Phytotoxicity on the foliage was noted for the AX formulations in this study. The foliage was noted to develop chlorotic and necrotic symptoms, especially at the margins, for both formulations. Symptoms were noted within a week of application. While the low rate seemed to have more chlorosis initially, the foliage seemed less injured following subsequent applications. In contrast, with the high rate, phytotoxic symptoms appeared to increase with subsequent applications.

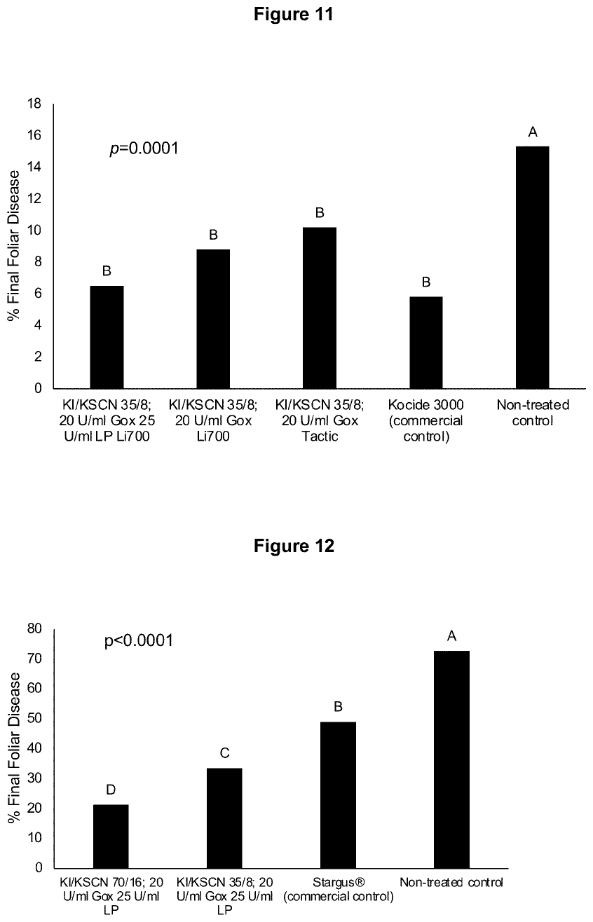

Data were analyzed with JMP-SAS for analysis of variance and significant differences, Tukey's HSD was used to assess multiple comparison of means (0.05) (FIG. 12).

### Example 8: Disease Suppression in Field Pumpkin

The AX Formulations showed efficacy against powdery mildew on pumpkins caused by Podosphaera xanthii. Transplants of pumpkin cultivar ‘Howden’ were planted into a Palmyra gravel loam using a complete randomized block design with four replicates. Plants were fertilized with 19-19-19 nitrogen, phosphorous and potassium (NPK) respectively at a rate of 240 pounds (lbs) of fertilizer per acre, and plants were hand-watered as needed. Plots were 25 feet long and rows were 7 feet apart. Plots consisted of 10 plants, and all data were collected from the center 5 plants. Regalia® (ProFarm Group, CA), a biological standard, and a non-treated control were included in the trial.

The products were applied using a CO₂ hand-held pressurized sprayer, that was calibrated to deliver 50 gallons per acre (gpa) at 42 psi. The spray boom consisted of 2 hollow cone nozzles (=TeeJet TXVS18) set on the 18 inch center. The products were applied after the first lesions of powdery mildew were visible on days 28, 36, 43 and 49 post-planting. On day 49 post-planting, the rates were lowered due to phytotoxicity.

Plants were rated for the percent leaf area with symptoms of powdery mildew. Ratings on days 34 and 44 post-planting were from the upper surface of the leaves, and on Day 43 post-planting, powdery mildew was rated on both the upper and lower leaf surface.

### Formulations

70 mM KI 16 mM KSCN; 20 U/ml Gox 25 U/ml LP
35 mM KI 8 mM KSCN; 20 U/ml Gox 25 U/ml LP

On all rating dates, both AX formulations and Regalia® reduced the severity of powdery mildew. Powdery mildew with the higher KI/KSCN rate (70/16) formulation treatment was consistently comparable or less than the Regalia® standard. The lower KI/KSCN (38/8) rate was less effective at reducing powdery mildew compared to the higher rate, but always substantially less than the non-treated control. The final rating (Day 43 post-planting) for the lower rate showed a reduced powdery mildew comparable to the biological standard, Regalia®.

Phytotoxicity on the foliage was noted for the formulations in this study. The foliage developed chlorotic and necrotic symptoms, especially at the margins, for both formulations. Symptoms were noted within a week of application. While the lower rate seemed to have more chlorosis initially, the foliage seemed less injured following subsequent applications. Whereas phytotoxic symptoms appeared to increase with subsequent applications of the higher rate. Symptoms were marginally acceptable with the low rate, but commercially unacceptable with the high rate. These data indicated that formulation is critical for effective disease suppressive activity. High salt formulations cause significant phytotoxicity.

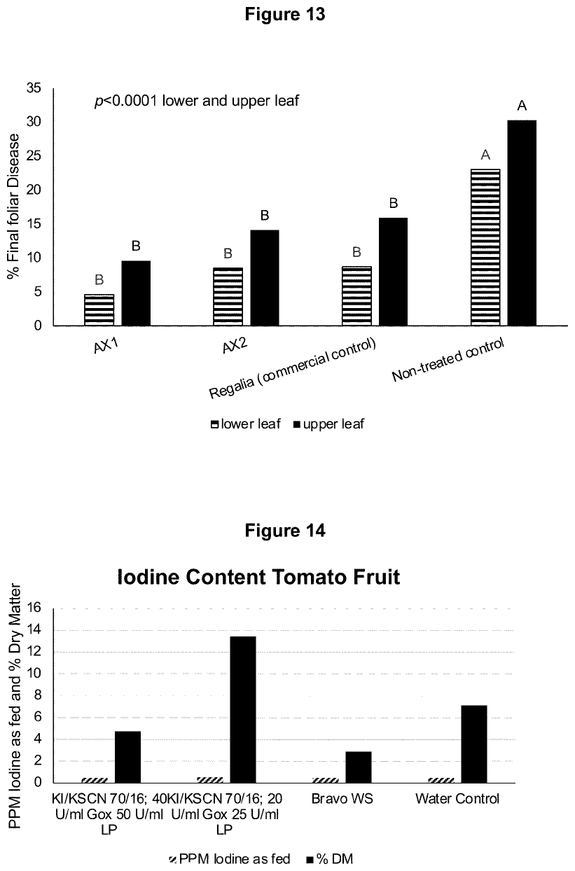

Data were analyzed with JMP-SAS for analysis of variance and significant differences, Tukey's HSD was used to assess multiple comparison of means (0.05) (FIG. 13).

### Example 9: AX Formulation and Dosing for Field Snap Beans

The AX Formulations were evaluated against white mold in snap beans caused by Sclerotinia sclerotiorum. The snap beans were planted in a Honeoye silt loam soil. The Huntington seed variant was planted with a Monosem planter at a rate of 9 seeds/ft (1⅜ inch spacing within rows) and 30 inch spacing between rows. Fertilizer (300 1b/A 15 N: 5 P: 10 K) was broadcast applied prior to planting and banded (350 1b/A) at planting. The pre-emergent herbicide, Dual Magnum® (1.8 pt/A) was applied on the same day for weed control. The growth stage of the snap beans at critical points was recorded to schedule fungicide applications and S. sclerotiorum inoculations.

The trial was a completely randomized block with four replications of each treatment and a nontreated control. Each plot was 10 feet long×2 rows wide. Two non-inoculated and nontreated rows separated plots between blocks and 5-foot sections separated plots within rows. Products were applied with a carbon dioxide-pressurized backpack sprayer with a volume of 27.4 gallons/Acre using a 38-inch-long boom using three flat fan TJ 8002VS nozzles spaced 19 inches apart on day 42 post-planting (approximately 10% of plants with at least one open flower) and day 49 post-planting (approximately 100% of plants with at least one open flower) with the second application at a lower rate (reduced by half) due to high phytotoxicity.

### Formulations

35 mM KI 8 mM KSCN; 20 U/ml Gox 25 U/ml LP
35 mM KI 8 mM KSCN; 20 U/ml Gox 12 U/ml LP
18 mM KI 4 mM KSCN; 10 U/ml Gox 12 U/ml LP
18 mM KI 4 mM KSCN; 10 U/ml Gox 6 U/ml LP

Inoculation with S. sclerotiorum ascospores led to a high incidence of white mold in nontreated plots. Both AX formulations resulted in substantial phytotoxicity (defoliation, stem burn-down, and complete loss of pods) which drifted to surrounding buffer areas. Disease control could not be evaluated. A single application of the AX formulations resulted in complete crop loss demonstrating that the correct formulation and dose are required for effective control of plant pathogens on crops.

### Example 10: Iodine Biofortification of Field Crops

Tomato. The AX Formulations were tested for the accumulation of iodide in tomato plant tissue. The tomato transplant cultivar ‘Mt. Fresh’ was planted using a complete randomized block design with four replicates into a Howard gravelly loam soil. Fertilizer 13-13-13 was applied at a rate of 1150 lbs per acre. Tomato herbicide, Sandea® (0.5 oz./a), Dual II Magnum® (1 pt./a), or Metribuzin DF® (0.33 lb./a) was applied preplan, 2 days before planting, over open furrows with a back-pack sprayer. Plants were hand-watered as needed with overhead irrigation. Bravos WS® (Syngenta, NC) was included as a standard commercial control:

### Formulations

70 mM KI 16 mM KSCN; 40 U/ml Gox 50 U/ml LP
70 mM KI 16 mM KSCN; 20 U/ml Gox 25 U/ml LP
Bravo WS 1.5 pt/acre

AX formulations were applied at a rate of 50 gpa to 4 replicates of tomatoes (2 tomato plants per each replicate) with a CO₂ hand-held pressurized sprayer (37 psi) on days 33, 40, 48, 54, and 62 days post-planting.

Tomato fruit was harvested on day 84 post-planting. Five tomatoes were randomly sampled from each of four replicates. Samples were submitted to iodine analysis (FIG. 14). Feed iodine analysis was performed by inductively-coupled plasma mass spectrometry (ICP-MS) in accordance with a modified version of the method for the determination of extractable iodine content in feed using ICP-MS. (Verband Deutscher Landwirtschaftlicher Untersuchiungs-und Forschungstalten (VDLUFA, 2006) Method book volume 3 (11.7.1) and Volume 7 Environmental analysis (2.2.2.3), incorporated by reference herein in its entirety). In brief, a solution of 7% ammonium hydroxide was added to 1 gram of feed to a final sample weight of 50 grams and subsequently incubated at 95° C. for one hour. Upon completion of the digestion, samples were sonicated for one additional hour at room temperature, centrifuged to pellet debris, and an aliquot was diluted for analysis on an Agilent Technologies 7900 ICP-MS. The ICP-MS was tuned to yield a minimum of 7500 cps sensitivity for 1 ppb yttrium (mass 89). The analysis was performed in Helium mode to reduce spectral interference and was tuned to limit to less than 1.0% oxide as determined by the 156/140 mass ratio and less than 2.0% double charged ions as determined by the 70/140 mass ratio.

This data showed greater iodine amounts in tomato fruit after application of AX formulations when compared with the water and fungicide (Bravo WS® (Syngenta, NC)) controls. This showed a biofortification of iodine in tomato fruit.

Potato. The AX formulation showed accumulation of iodide in potato and tomato leaves. The Atlantic potato cultivar was planted using a complete randomized block design with four replicates into a Howard gravelly loam soil. Furrows were opened and 13-13-13 nitrogen, phosphorous, and potassium fertilizer was applied at a rate of 150 lbs./acre with a potato planter. Potatoes were planted in a randomized complete block design with four replicates. The herbicides Lorox® (2 1b./a) and Dual II Magnum® were applied 4 days after planting at a rate of 1 pint/acre. Bravos WS® (Syngenta, NC) was included as a standard commercial pesticide control.

### Formulations

35 mM KI 8 mM KSCN; 40 U/ml Gox 50 U/ml LP
35 mM KI 8 mM KSCN; 20 U/ml Gox 25 U/ml LP

AX formulations were applied at a rate of 50 gpa to 4 replicates (5 potato plants each replicate) of potatoes with a CO₂ hand-held pressurized sprayer (37 psi) on days 41, 48, 56 and 63 days post-planting.

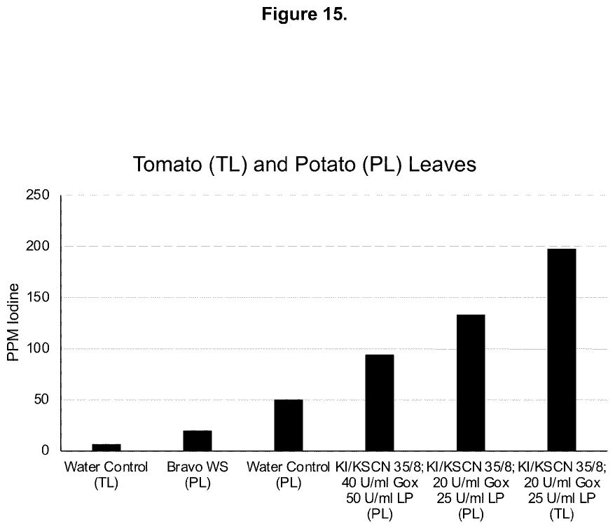

Potato leaves were harvested on day 82 post-planting. Five leaflets were randomly sampled from each of four replicates. Samples were submitted to iodine analysis as disclosed above. As a control for potato leaf tissue analysis, an AX formulation (35 mM KI 8 mM KSCN; 20 U/ml Gox 25 U/ml LP) was applied (10 ml hand sprayed) to a tomato plant (Mt. Fresh cultivar). Leaves were collected after 24 h and submitted with the potato leaf tissue for iodine analysis. This data showed greater iodine amounts in potato and tomato leaves after application of AX formulations when compared with the water and fungicide (Bravo WS® (Syngenta, NC)) controls. This showed a biofortification of iodine in potatoes and tomatoes. (FIG. 15).

### Example 11: Disease Eradication on Corn Salad Seeds

The following utilized Formulation C. AX was evaluated for the eradication of seedborne fungi and bacteria corn salad (Valerianella locusta). Seeds were infested by Phoma valerianellae, Acidovorax spp., and other microbial pathogens. 1 g of seeds was poured into 15 mL of each of four treatments: 5 AX formulations and 1% bleach in sterile DI water.

### Formulations

35 mM KI, 8 mM KSCN, 300 mM Glucose, 200 mM Sodium Acetate, 20 U/mL Glucose-Oxidase, 25 U/mL Lactoperoxidase
35 mM KI, 4 mM KSCN, 300 mM Glucose, 200 mM Sodium Acetate, 20 U/mL Glucose-Oxidase, 25 U/mL Lactoperoxidase
35 mM KI, 0 mM KSCN, 300 mM Glucose, 200 mM Sodium Acetate, 20 U/mL Glucose-Oxidase, 25 U/mL Lactoperoxidase
35 mM KI, 8 mM KSCN, 150 mM Glucose, 200 mM Sodium Acetate, 20 U/mL Glucose-Oxidase, 25 U/mL Lactoperoxidase
35 mM KI, 8 mM KSCN, 75 mM Glucose, 200 mM Sodium Acetate, 20 U/mL Glucose-Oxidase, 25 U/mL Lactoperoxidase

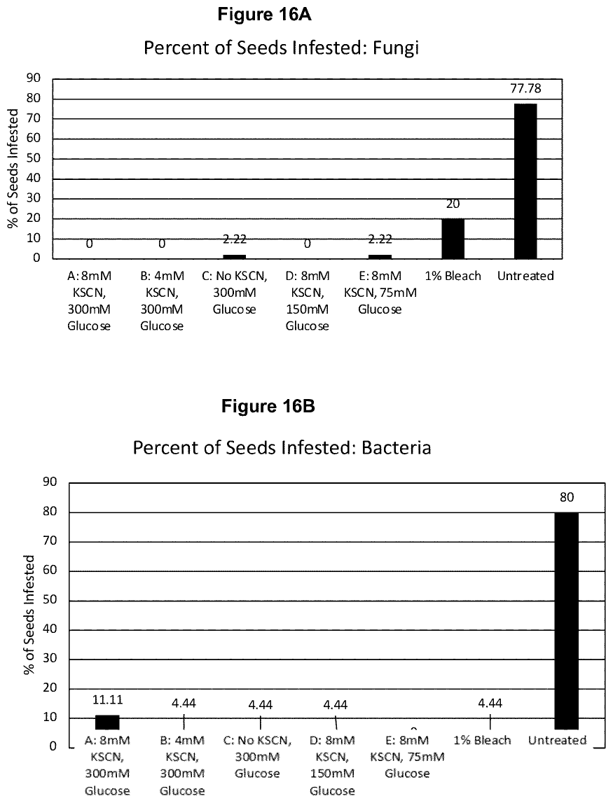

1 g of seeds was also set aside as a dry untreated control. The seeds were washed in 15 ml of AX formulations or 1% bleach in a 50 mL Erlenmeyer and placed on a shaker for 30 minutes. The seeds were removed from the washing treatments and rinsed with sterile DI water, then left overnight to dry in a fume hood. Once dry, seeds were placed on two media types: YDC (Yeast extract-dextrose-CaCO3; see below for recipe), which favors the growth of bacteria, and PDA (Potato Dextrose Agar; see below for recipe) with added antibiotics which favor the growth of fungi. For each treatment, three plates of each media type held 15 seeds each for a total of 45 seeds. After two days, on day 3, the number of seeds which were surrounded by or covered in non-seed growth (i.e., bacterial mat or fungal hyphae) were counted. Growth on seeds on YDC media was considered evidence of surviving bacteria infesting the seed surface, and growth on seeds on PDA was considered evidence of fungal infestation of the seed surface. The percentage of seeds infested in each category is shown in FIG. 16A & FIG. 16B.

Yeast Extract Dextrose-CaCO₃ (YDC) Media was prepared by combining 20 g/L Glucose, 10 g/L Yeast Extract, 20 g/L CaCO₃, 15 g/L Agar with DI water and autoclaving.

Potato Dextrose Agar (PDA) Media (Becton, Dickinson & Company, Sparks, MD 21152 USA). 39 g/L added to DI water and autoclaved according to instructions. 100 μg/mL Rifamycin, 50 μg/mL Polymyxin B, 500 μg/mL Ampicillin, 50 μg/mL Vancomycin added after autoclaving.

All AX formulations exceeded the antifungal control of the 1% bleach solution. Furthermore, the formulation without KSCN show nearly identical control of fungi (FIG. 16A). KSCN was therefore not alone responsible for the eradication of fungi associated with corn salad seeds.

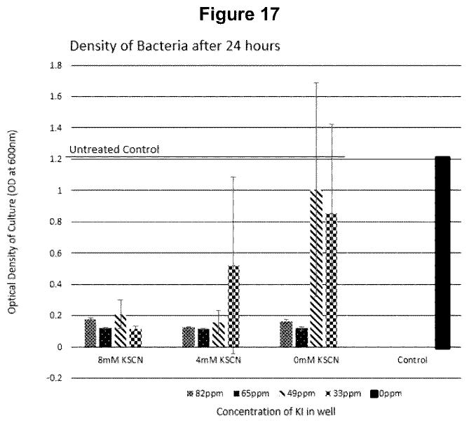

Most AX formulations equaled the antibacterial eradication efficacy of 1% bleach (FIG. 17B). The formulation without KSCN (0 mM KSCN) eradicated seed-born bacteria as well or better than those formulations with KSCN. Interestingly, a formulation with lower glucose (75 mM) fully eradicated seed-born bacteria.

### Example 12: Bacterial Growth Inhibition in Nutrient Broth

The following utilized Formulation C. AX was evaluated for inhibition of Pseudomonas syringae growth in nutrient broth suspension:

### Formulations

35 mM KI, 8 mM KSCN, 300 mM Glucose, 200 mM Sodium Acetate, 20 U/mL Glucose-Oxidase, 25 U/mL Lactoperoxidase
35 mM KI, 4 mM KSCN, 300 mM Glucose, 200 mM Sodium Acetate, 20 U/mL Glucose-Oxidase, 25 U/mL Lactoperoxidase
35 mM KI, 0 mM KSCN, 300 mM Glucose, 200 mM Sodium Acetate, 20 U/mL Glucose-Oxidase, 25 U/mL Lactoperoxidase

On Day 0, suspensions of Pseudomonas syringae in nutrient broth (NB; 5 g/L Protease Peptone, 5 g/L NaCl, 2 g/L Yeast Extract to DI water) were prepared using mass from a bacteria mat of isolate NYT1 maintained on YDC media (Yeast Extract Dextrose-CaCO₃: 20 g/L Glucose, 10 g/L Yeast Extract, 20 g/L CaCO₃, 15 g/L Agar, DI water) and incubated at 30° Celsius. On day 1, The optical density at 600 nm (OD600) of the suspension was measured using a Synergy H1 microplate reader. This was diluted to an approximate OD600 of 0.14 in 700 μL volumes in the wells of three 48 well plates. For each formulation, four doses were tested in triplicate: 10 μL, 8 μL, 6 μL, and 4 μL (82 ppm, 65 ppm, 49 ppm, and 33 ppm KI) which were added to three wells each per treatment. The plates were then incubated for 20 hours at 30° Celsius. On day 2, the OD600 of each well was measured. Controls with no added treatments and the average of the three replicates is shown in FIG. 17. Higher OD600 was used as evidence of growth of the suspension culture while low OD at or below the initial 0.14 was considered evidence of inhibited growth.

The eradication of Pseudomonas syringae bacteria in nutrient broth was equally effective with and without KSCN at the two higher formulations (82 pmm, 65 ppm). KSCN enhanced antibacterial efficacy at the two lower formulations (49 ppm, 33 ppm).

### Example 12: Fungal Growth Inhibition on Media Plates

The following utilized Formulation C. AX formulations were evaluated for inhibition of Botrytis cinerea Growth:

### Formulations

35 mM KI, 8 mM KSCN, 300 mM Glucose, 200 mM Sodium Acetate, 20 U/mL Glucose-Oxidase, 25 U/mL Lactoperoxidase
35 mM KI, 2 mM KSCN, 300 mM Glucose, 200 mM Sodium Acetate, 20 U/mL Glucose-Oxidase, 25 U/mL Lactoperoxidase
35 mM KI, 0 mM KSCN, 300 mM Glucose, 200 mM Sodium Acetate, 20 U/mL Glucose-Oxidase, 25 U/mL Lactoperoxidase

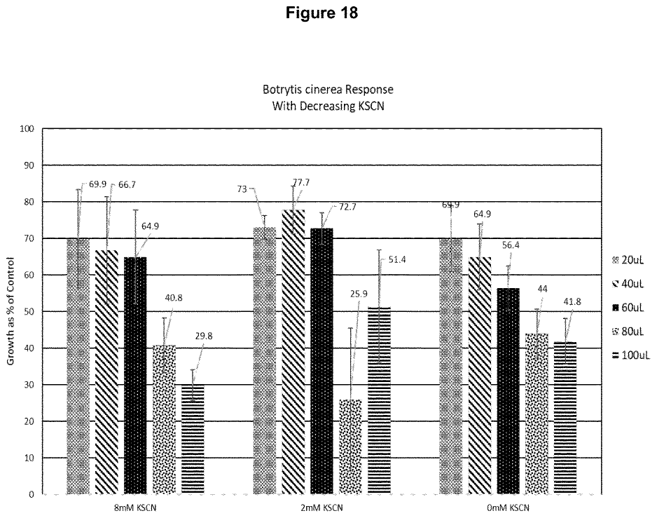

Fungi were grown on Potato Fructose Agar (PFA; 25 g/L Fructose, 4 g/L Yeast Extract, 4 g/L Potato Starch, and 15 g/L Agarose) plates. A sample of Botrytis cinerea isolate was transferred to a new 100 mm PFA plate on day 1 in preparation for the experiment. On day 9, five volumes of each treatment, 100 μL, 80 μL, 60 μL, 40 μL, and 20 μL, were pipetted onto three 55 mm PFA media plates per dose and spread across the surface with an inoculation loop. A cork borer was used to remove 5 mm diameter plugs from the Botrytis cinerea mycelium, and a single plug was placed in the center of each 55 mm PFA plate. Three additional untreated PFA plates were prepared with mycelia plugs, as controls. On day 12, the radius of the mycelium on each plate was measured with electronic calipers. The average radius of growth on plates for dose of each treatment was graphed as a percentage of the average radius of the control plates (FIG. 18).

The eradication of the fungus Botrytis cinerea on media plates by AX formulations was dose dependent. The efficacy was not dependent on KSCN. AX formulations without KSCN (0 mM KSCN) were slightly less efficacious than those with KSCN (8 mM KSCN).

### Example 13: Reagents and Materials

The following chemicals reagents were used to synthesize the biocidal agents: Potassium Iodide (KI, Deepwater Chemicals, 101.24, CAS No. 7681-11-0), Potassium Thiocyanate (KSCN, Fisher Scientific, AC196585000, CAS No. 333-20-0), Glucose (D-Glucose, 99%, anhydrous, Acros Organics, 10032070, CAS No. 50-99-7), Sodium Phosphate Monobasic (Sodium Phosphate Monobasic Anhydrous, VWR Life Sciences, 0571-2.5 KG, CAS No. 7558-80-7), Sodium Phosphate Dibasic (Sodium Phosphate Dibasic Anhydrous, VWR Life Science, 0404-1 KG, CAS No. 7558-79-4), pressurized oxygen (Airgas OX 200), and hematite scaffold (average particle size, 40 μm, Powdertech, Rhosmedre, Wrexham, United Kingdome). All water was obtained from an Express Water RO System water purifier (Express Water, RODI10-D).

Bovine milk enzyme Lactoperoxidase (LP, E.C. 1.11.1.7) was purchased from Tatua Dairy Company (Morrinsville, New Zealand). Fungal enzyme Glucose Oxidase (GOx, β-D-Glucose: Oxygen 1-oxidoreductase, Source: Aspergillus niger, E.C. 1.1.3.4) was purchased from Millipore Sigma.

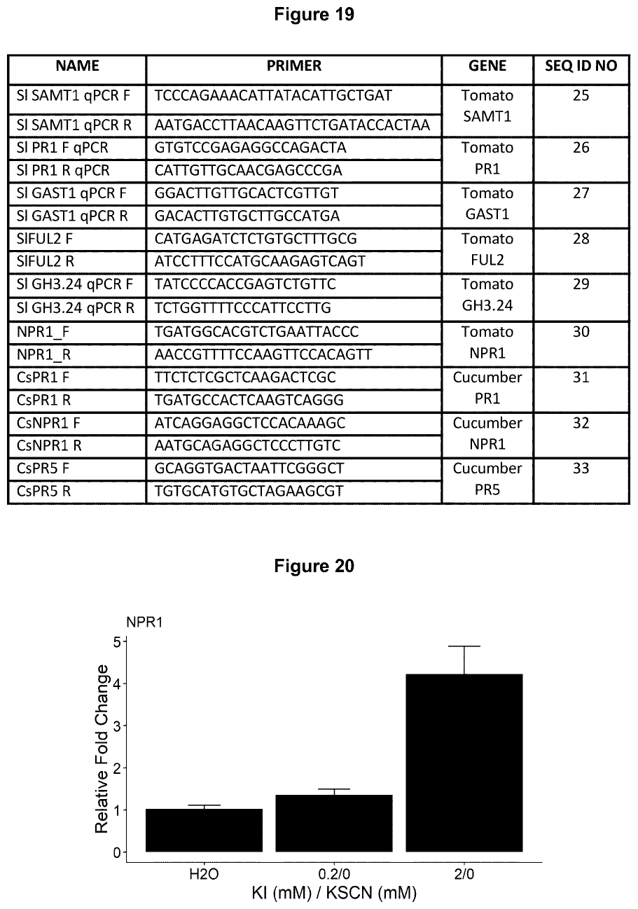

For qPCR analysis the following materials and reagents were used: NEB Monarch Total RNA Miniprep Kit (T2010S), NEB Luna Universal One-Step RT-qPCR Kit (E3005S), qPCR primers from FIG. 19.

### Example 14: Formulations

### Formulation E: Production of AX SAR and Biocidal Agents

An AX SAR and Biocidal formulation (4 mM KI, 0.8 mM KSCN, 50 mM Glucose, and 10 mM Sodium Phosphate, pH 7.0, 10 U/mL Glucose Oxidase, and 12 U/mL Lactoperoxidase) was produced from a 1× salt-sugar solution and subsequent addition of the two enzyme solutions.

A 136× (10 mg/ml, 1640 U/ml) solution of LP (Lactoperoxidase, from bovine milk, activity 164 U/mg) was prepared in 25 mM Phosphate Buffer at pH 7.0 by addition of 7.3 mL of 25 mM Phosphate Buffer (pH 7.0) to 73 mg of LP. The LP solution was stored in a 4° C. refrigerator until use.

A 160× (10 mg/ml, 1600 U/ml) solution of GOx (Glucose Oxidase, from Aspergillus niger, activity 160 U/mg) was prepared by addition of 6.3 mL of Deionized water to 63 mg of GOx. The GOx solution was stored in a 4° C. refrigerator until use.

The AX biocidal formulation was produced by addition of 664 mg of Potassium Iodide, 77.7 mg of Potassium Thiocyanate, 9.0 g of D-Glucose, 507.5 mg of Sodium Phosphate Monobasic and 819.1 mg of Sodium Phosphate Dibasic to a 2 L media bottle equipped with a magnetic stir bar. To this, 986 mL of DI water was added, and the mixture was stirred until homogeneous. The pH was measured to be 7.0. Next, 7.3 mL of the 136× LP solution was added to the stirring solution followed by addition of 6.3 mL of the 160× GOx solution. The bottle was closed firmly, and the reaction mixture was stirred for 1 hour at 25° C. using a magnetic stirrer. After 1 hour, the solution reached a pH of 6.8.

Formulation F: Production of AX Biocidal Agent with Enzymes without KSCN

An AX Biocidal formulation (4 mM KI, 50 mM Glucose, and 10 mM Sodium Phosphate, pH 7.0, 10 U/mL Glucose Oxidase, and 12 U/mL Lactoperoxidase) was produced from a 1× salt-sugar solution and subsequent addition of the two enzyme solutions.

A 136× (10 mg/ml, 1640 U/ml) solution of LP (Lactoperoxidase, from bovine milk, activity 164 U/mg) was prepared in 25 mM Phosphate Buffer at pH 7.0 by addition of 7.3 mL of 25 mM Phosphate Buffer (pH 7.0) to 73 mg of LP. The LP solution was stored in a 4° C. refrigerator until use.

A 160× (10 mg/ml, 1600 U/ml) solution of GOx (Glucose Oxidase, from Aspergillus niger, activity 160 U/mg) was prepared by addition of 6.3 mL of Deionized water to 63 mg of GOx. The GOx solution was stored in a 4° C. refrigerator until use.

The AX biocidal formulation was produced by addition of 664 mg of Potassium Iodide, 9.0 g of D-Glucose, 507.5 mg of Sodium Phosphate Monobasic and 819.1 mg of Sodium Phosphate Dibasic to a 2 L media bottle equipped with a magnetic stir bar. To this, 986 mL of DI water was added, and the mixture was stirred until homogeneous. The pH was measured to be 7.0. Next, 7.3 mL of the 136× LP solution was added to the stirring solution followed by addition of 6.3 mL of the 160× GOx solution. The bottle was closed firmly and the reaction mixture was stirred for 1 hour at 25° C. using a magnetic stirrer. After 1 hour, the solution reached a pH of 6.8.

Formulation G: Production of AX SAR and Biocidal Agents without LP

An AX SAR and Biocidal formulation (4 mM KI, 0.8 mM KSCN, 50 mM Glucose, and 10 mM Sodium Phosphate, pH 7.0, and 10 U/mL Glucose Oxidase) was produced from a 1× salt-sugar solution and subsequent addition of the GOx enzyme solutions.

A 160× (10 mg/ml, 1600 U/ml) solution of GOx (Glucose Oxidase, from Aspergillus niger, activity 160 U/mg) was prepared by addition of 6.3 mL of Deionized water to 63 mg of GOx. The GOx solution was stored in a 4° C. refrigerator until use.

The AX biocidal formulation was produced by addition of 664 mg of Potassium Iodide, 77.7 mg of Potassium Thiocyanate, 9.0 g of D-Glucose, 507.5 mg of Sodium Phosphate Monobasic and 819.1 mg of Sodium Phosphate Dibasic to a 2 L media bottle equipped with a magnetic stir bar. To this, 986 mL of DI water was added, and the mixture was stirred until homogeneous. The pH was measured to be 7.0. Next, 6.3 mL of the 160× GOx solution. The bottle was closed firmly, and the reaction mixture was stirred for 1 hour at 25° C. using a magnetic stirrer. After 1 hour, the solution reached a pH of 6.8.

### Formulation H: Production of AX Biocidal Agents in the Field at Scale

An AX Biocidal formulation (4 mM KI, 0.8 mM KSCN, 50 mM Glucose, 10 mM Sodium Phosphate, pH 7.0, 10 U/mL Glucose Oxidase, and 12 U/mL Lactoperoxidase) was produced from a 28× salt-sugar solution and subsequent addition of the two enzyme solutions.

A 136× (10 mg/ml, 1640 U/ml) solution of LP (Lactoperoxidase, from bovine milk, activity 164 U/mg) was prepared in 25 mM Phosphate Buffer at pH 7.0 by addition of 415 mL of 25 mM Phosphate Buffer (pH 7.0) to 4.15 g of LP. The LP solution was stored in a 4° C. refrigerator until use.

A 160× (10 mg/ml, 1600 U/ml) solution of GOx (Glucose Oxidase, from Aspergillus niger, activity 160 U/mg) was prepared by addition of 355 mL of Deionized water to 3.55 g of GOx. The GOx solution was stored in a 4° C. refrigerator until use.

A 28× solution of KI, KSCN, Glucose, and Sodium Phosphate buffer was prepared by addition of 37.6 g of Potassium Iodide, 4.4 g of Potassium Thiocyanate, 510.8 g of D-Glucose, 28.8 g of Sodium Phosphate Monobasic and 46.4 g of Sodium Phosphate Dibasic to a 2 L media bottle equipped with a magnetic stir bar. To this, 2 L of DI water was added, and the mixture was heated at 80° C. and stirred until homogeneous. The pH was measured to be 7.0.

In a 30-gallon tractor tank, 15 gallons of water was added along with the 28× sugar-salt solution. Next, the 415 mL of 136× LP solution was added followed by the 160× GOx solution. The solution was mixed with a tractor recirculation system for 15 minutes before it was sprayed on plants.

Formulation I: Production of AX Biocidal Agent with Enzymes without KSCN in the Field at Scale

An AX Biocidal formulation (4 mM KI, 50 mM Glucose, 10 mM Sodium Phosphate, pH 7.0, 10 U/mL Glucose Oxidase, and 12 U/mL Lactoperoxidase) was produced from a 28× salt-sugar solution and subsequent addition of the two enzyme solutions.

A 136× (10 mg/ml, 1640 U/ml) solution of LP (Lactoperoxidase, from bovine milk, activity 164 U/mg) was prepared in 25 mM Phosphate Buffer at pH 7.0 by addition of 415 mL of 25 mM Phosphate Buffer (pH 7.0) to 4.15 g of LP. The LP solution was stored in a 4° C. refrigerator until use.

A 160× (10 mg/ml, 1600 U/ml) solution of GOx (Glucose Oxidase, from Aspergillus niger, activity 160 U/mg) was prepared by addition of 355 mL of Deionized water to 3.55 g of GOx. The GOx solution was stored in a 4° C. refrigerator until use.

A 28× solution of KI, Glucose, and Sodium Phosphate buffer was prepared by addition of 37.6 g of Potassium Iodide, 510.8 g of D-Glucose, 28.8 g of Sodium Phosphate Monobasic and 46.4 g of Sodium Phosphate Dibasic to a 2 L media bottle equipped with a magnetic stir bar. To this, 2 L of DI water was added, and the mixture was heated at 80° C. and stirred until homogeneous. The pH was measured to be 7.0.

In a 30-gallon tractor tank, 15 gallons of water was added along with the 28× sugar-salt solution. Next, the 415 mL of 136× LP solution was added followed by the 160× GOx solution. The solution was mixed with a tractor recirculation system for 15 minutes before it was sprayed on plants.

### Formulation J: Production of AX SAR and Biostimulant Agents

An AX SAR and Biostimulant formulation (0.2 or 2 mM KI, 50 mM Glucose, 10 mM Sodium Phosphate, pH 7.0, 2 or 5 U/mL Glucose Oxidase, and 3 or 6 U/mL Lactoperoxidase) was produced from a 1× salt-sugar solution and subsequent addition of the enzyme solutions.

Two 136× (10 mg/ml, 1640 U/ml) solutions of LP (Lactoperoxidase, from bovine milk, activity 164 U/mg) were prepared in 25 mM Phosphate Buffer at pH 7.0 by addition of either 0.35 mL of 25 mM Phosphate Buffer (pH 7.0) to 3.5 mg of LP or 0.17 mL of 25 mM Phosphate Buffer (pH 7.0) to 1.7 mg of LP. The LP solutions were stored in a 4° C. refrigerator until use.

Two 160× (10 mg/ml, 1600 U/ml) solutions of GOx (Glucose Oxidase, from Aspergillus niger, activity 160 U/mg) were prepared by addition of either 0.3 mL of Deionized water to 3 mg of GOx or 0.1 mL of Deionized water to 1.2 mg of GOx. The GOx solutions were stored in a 4° C. refrigerator until use.

The AX biostimulant formulations were prepared by addition of either 31.5 mg of Potassium Iodide (2 mM KI) or 3.2 mg of Potassium Iodide (0.2 mM KI), 855.8 mg of D-glucose, 48.2 mg of sodium phosphate monobasic, and 77.8 mg of sodium phosphate dibasic to a 125 mL bottle equipped with a magnetic stir bar. To this, 94 mL of DI water was added, and the solution was stirred until homogeneous. Next, either 0.35 mL (6 U/mL LP) or 0.17 mL (3 U/mL LP) of the 136× solution of LP was added, followed by 0.3 mL (5 U/mL GOx) or 0.1 mL (2 U/mL GOx) of the 160× solution of GOx solution. The bottle was closed firmly and the reaction mixture was stirred for 1 hour at 25° C. using a magnetic stirrer.

### Example 15: Measuring Systemic Acquired Response (SAR) Gene Activation

Two methods of application were used to measure SAR gene activation in response to AX formulations: For tomatoes, (1) 10 ml of AX was applied to each plant with a non-mechanized hand sprayer and 2) for cucumbers, AX was applied at a rate of 25 gpa (gallons per acre) with a hand-held pressurized CO₂ sprayer at 37 psi.

For tomato plants, two leaflets were sampled from the second-youngest leaf. For cucumber plants, the second youngest leaf was sampled as a single biological replicate. Leaf tissue was homogenized via mortar and pestle in QIAGEN RLT buffer, with β-mercaptoethanol at a 1:100 dilution. Total RNA was extracted from homogenized leaf tissue with the Monarch Total RNA Miniprep Kit (New England Biolabs (NEB), Cat. No. T2010S) following standard manufacturer protocols. Total RNA concentration was determined with a Nanodrop spectrophotometer.

Gene expression was determined through Reverse Transcription-quantitative Polymerase Chain Reaction (RT-qPCR) with the LUNA Universal One-Step RT-qPCR Kit (NEB Cat. No. E3005L) following manufacturer protocols. RT-qPCR was run on a QuantStudio 7 Pro, and the resulting data was analyzed via the 2-delta CT method in the Design and Analysis App (DA2) from ThermoFisher Scientific. Relative fold-change compared the expression of target genes to an internal control gene, Tubulin for tomato and

Actin-2 for cucumber, within each group. The relative changes between the target and control genes between a given treatment group and the water control were. Primers are shown in FIG. 19.

### Example 16: Inducing Systemic Acquired Response (SAR) Gene Activation

Tomato. For tomato, 10 ml of AX formulations were applied to six-week-old plants, (cultivar Mt. Fresh), sown and germinated in Cornell potting mix. The foliar applications were applied uniformly to leaf tissue. One day post-application, samples were taken and analyzed for (SAR) marker genes, NONEXPRESSER OF PR GENES 1 (NPR1), PATHOGENESIS-RELATED 1 (PR1), and SALICYLIC ACID METHYLTRANSFERASE 1 (SAMT1) gene expression. Leaves sprayed with water were used as controls.

All AX formulations were made using the production method described in Formulation J. The formulations were as follows:

### Formulations

0.2 mM KI, 50 mM glucose, 10 mM phosphate, 2 U/ml GOx, 3 U/ml LP
2 mM KI, 50 mM glucose, 10 mM phosphate, 5 U/ml GOx, 6 U/ml LP

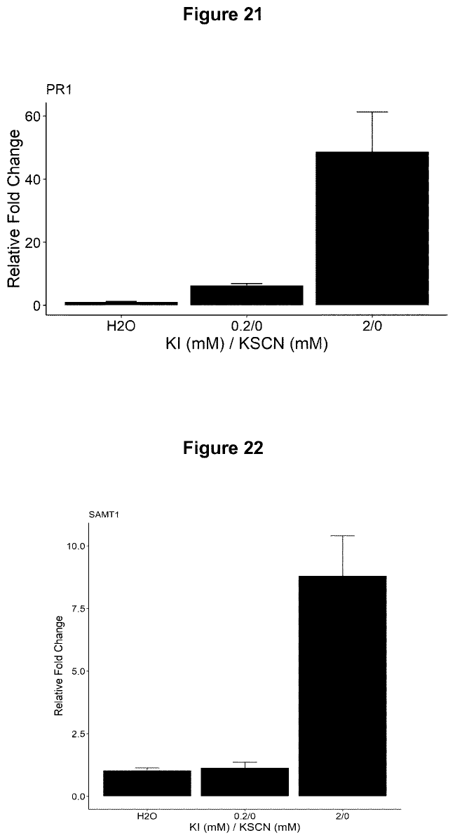

Both AX formulations upregulated NPR1 (1.4 and 4.2 relative fold-change), PR1 (6.3 and 48.7 relative fold change), and SAMT1 (1.1 and 8.8 relative fold-change). The 2 mM KI treatment induced significantly higher gene expression than the 0.2 mM KI formulation (FIGS. 20-22). These data show that KSCN is not required for induction of SAR marker genes.

Cucumber. For cucumber, seeds were germinated in Cornell potting mix and grown in the greenhouse for 4 weeks and subsequently transferred to the field for foliar applications. The AX formulations were applied at a rate of 25 gallons per acre (gpa) with a CO₂ hand-held pressurized (37 psi) sprayer. The spray boom consisted of 2 hollow cone nozzles (=TeeJet TXVS18) set on the 18-inch center. One day post-application, samples were taken and analyzed for upregulation of Systemic Acquired Resistance (SAR) marker genes, Nonexpresser of PR Genes 1 (NPR1), Pathogenesis-Related 1 (PR1) and Pathogenesis-Related 5 (PR5) in cucumber plants. Leaves sprayed with water were used as controls.

All AX formulations were made using the production method described in Formulation E and G:

### Formulations

4/0.8 mM KI/KSCN, 50 mM glucose, 10 mM phosphate, 10 U/ml GOx, 12 U/ml LP
4/0.8 mM KI/KSCN, 50 mM glucose, 10 mM phosphate, 12 U/ml GOx

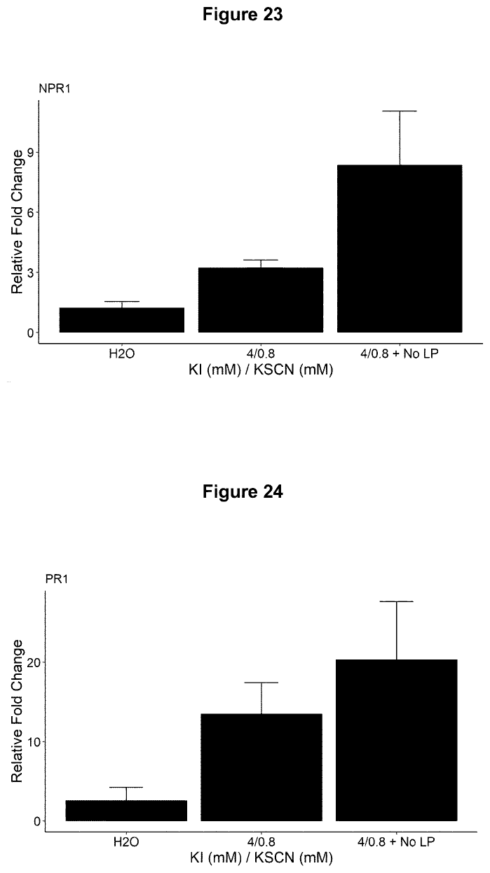

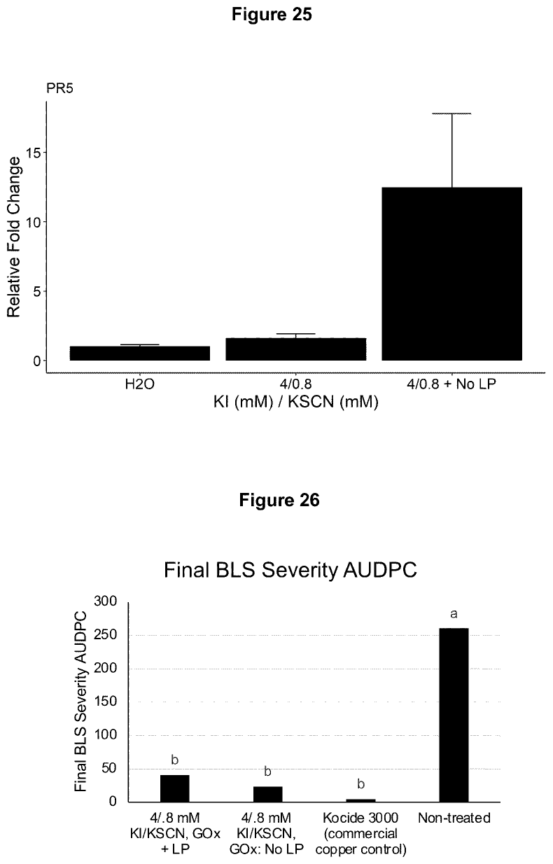

Both AX formulations with and without lactoperoxidase (LP) upregulated NPR1 (3.3 and 8.4 relative fold-change), PR1 (13.5 and 20.4 relative fold-change), and PR5, (1.6 and 12.5 fold-change). Surprisingly, the treatments with no LP induced significantly higher gene expression than the formulation with LP. (FIGS. 23-25.)

### Example 17: Disease Suppression in Beets BLS

Bacterial leaf spot (BLS), caused by the bacterium, Pseudomonas syringae pv. aptata, is a major constraint to table beet production. A replicated small plot trial was conducted to evaluate selected pesticides for BLS control in table beets. The experimental design of the trial was a completely randomized block with four replications of each treatment and a nontreated control. Treatments were applied with a carbon dioxide-pressurized backpack sprayer with a volume of 26.4 gallons/A (30 psi) using a 38-inch-long boom using four flat fan TJ 8002VS nozzles spaced 19 inches apart. Pesticides were applied at 44, 52, and 58 days after planting (DAP). The trial was inoculated with P. syringae pv. aptata at 48 DAP. The selected AX formulations were also compared to the current industry commercial standard, Kocide 3000-O (copper hydroxide) and a nontreated control. The area under the disease progress curve (AUDPC) was calculated to quantify epidemic progress within each plot. The effect of fungicides on epidemic progress (AUDPC) was analyzed using analysis of variance (ANOVA). A Fisher's least significant difference (LSD) test at P=0.05 was used to separate the means. All analysis were performed in the statistical software, Genstat Version 22 (Hemel Hempstead, UK).

All AX formulations were made using the production method described in Formulation E and G. The formulations were as follows:

### Formulations

4/0.8 mM KI/KSCN, 50 mM glucose, 10 mM phosphate, 10 U/ml GOx, 12 U/ml LP
4/0.8 mM KI/KSCN, 50 mM glucose, 10 mM phosphate, 10 U/ml GOx

Both AX treatments, with and without lactoperoxidase (LP), provided control of BLS, and for Area Under Disease Progress Curve (AUDPC), were statistically equivalent to Kocide 3000-O. Beet foliage showed no phytotoxicity from AX formulations in this study. The AX formulations prepared with only GOx were about as effective as the Gox+LP. This surprisingly indicated that LP is not required for disease suppressive activity. (FIG. 26.)

### Example 18: Disease Suppression of Cercospora Leaf Spot of Beets

Cercospora leaf spot (CLS), caused by the fungus, Cercospora beticola, is a major pathogen for table beets and sugar beets worldwide. C. beticola can rapidly develop resistance to fungicides with a single-site mode of action. A replicated small plot trial was conducted to evaluate selected fungicides for CLS control in table beets. The trial was a completely randomized block design with four replications of each treatment and a nontreated control. Treatments were applied with a carbon dioxide-pressurized backpack sprayer with a volume of 26.4 gallons/A (30 psi) using a 38-inch-long boom using four flat fan TJ 8002VS nozzles spaced 19 inches apart. Commercial fungicide treatments were applied four times at 62, 68, 78, and 83 DAP. However, because there was a delay in availability of the AX treatments, the first application was made at 68 DAP. The remaining applications were made at 78, 83, and 92 DAP for a total of four applications. The trial was inoculated with a mycelial suspension of C. beticola at 63 DAP. The area under disease progress curve (AUDPC) was calculated to quantify epidemic progress within each plot. The effect of fungicides on final CLS severity and epidemic progress (AUDPC) were analyzed using analysis of variance (ANOVA). A Fisher's least significant difference (LSD) test at P=0.05 was used to separate the means. All analysis were performed in the statistical software, Genstat Version 22 (Hemel Hempstead, UK).

All AX formulations were made using the production method described in Formulation E and G. The formulations were as follows:

### Formulations

4/0.8 mM KI/KSCN, 50 mM glucose, 10 mM phosphate, 10 U/ml GOx, 12 U/ml LP 4/0.8 mM KI/KSCN, 50 mM glucose, 10 mM phosphate, 10 U/ml GOx

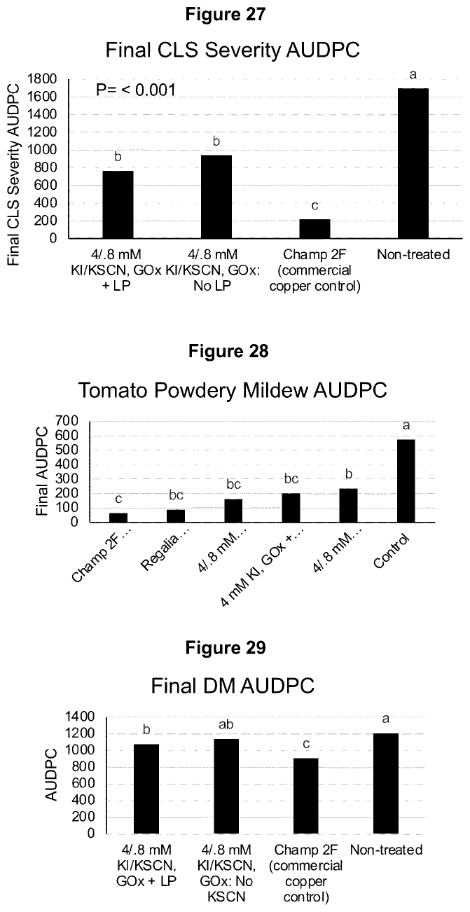

Despite being applied for the first time after the other treatments and inoculation with C. beticola, both AX treatments, with and without lactoperoxidase (LP), provided CLS control and were not significantly different from each other. The AX formulations prepared with only GOx were about as effective as the Gox+LP. This surprisingly indicated that LP is not required for disease suppressive activity. (FIG. 27.)

### Example 19: Disease Suppression of Powdery Mildew on Tomato

Powdery mildew of tomato is caused by the fungal pathogen, Oidium lycopersicum. It is an emerging disease which has become more problematic in greenhouse and field production of tomatoes. A replicated small plot trial was conducted to evaluate selected fungicides for control of powdery mildew on tomatoes. Tomato CV ‘Mountain Fresh” transplants (4 weeks old) were planted on Jul. 11, 2023. The trial was a completely randomized block design with four replications of two plant plots. Commercial fungicides, Champ 2F and Regalia, and AX treatments were applied five times at a rate of 25 gallons per acre (gpa) with a CO₂ hand-held pressurized (37 psi) sprayer 43, 52, 58, 65, and 71 DAP. The spray boom consisted of 2 hollow cone nozzles (=TeeJet TXVS18) set on the 18-inch center. Plants were rated for the percent leaf area with symptoms of powdery mildew on 50, 58, 64, and 72 DAP to track epidemic development. Plots were not inoculated; infection was induced from natural field inoculum. The area under disease progress curve (AUDPC) was calculated to quantify epidemic progress within each plot. AUDPC data were analyzed with JMP-SAS for analysis of variance and significant differences, Tukey's HSD was used to assess multiple comparison of means (0.05).

All AX formulations were made using the production method described in Formulation E, F, and G. The formulations were as follows:

### Formulations

4/0.8 mM KI/KSCN, 50 mM glucose, 10 mM phosphate, 10 U/ml GOx, 12 U/ml LP
4/0.8 mM KI/KSCN, 50 mM glucose, 10 mM phosphate, 10 U/ml GOx
4.0 mM KI, 50 mM glucose, 10 mM phosphate, 10 U/ml GOx, 12 U/ml LP

All three AX treatments reduced powdery mildew disease severity and slowed epidemic progress when compared to the control and were not significantly different from each other. The AX treatment with both GOx and LP was slightly more effective than the treatments with GOx alone or with KI alone. (FIG. 28.)

### Example 20: Disease Suppression of Downy Mildew of Cucumber

Downy mildew of cucumber caused by the oomycete pathogen, Pseudoperonospra cubensis, is an extremely destructive pathogen. A replicated small plot trial was conducted to evaluate selected fungicides for downy mildew control on cucumbers. Cucumber CV “Marketmore 76” seeds were planted on Jun. 22, 2023. The trial was a completely randomized block design with four replications of each treatment and a nontreated control. Commercial fungicide and AX treatments were applied three times at a rate of 25 gallons per acre (gpa) with a Massey Furgeson 231 tractor using a 30-gallon cone tank and spray boom fitted with four TXVS-18 hollow cone jet nozzles. The tractor was operating in 4^{th} gear at 1700 rpm to achieve the desired speed of 3.2 mph for an application rate of 25 gpa with a pressure of 40 psi. 70, 76, and 84 DAP.

Plants were rated for the percent leaf area with symptoms of downy mildew on 77, 83, and 91 DAP. All ratings were from evaluating the upper leaf surface for the percent leaf area with symptoms of downy mildew. A single rating was recorded from each of 5 plants within the center of each plot. On each date, the foliage was rated for the percent leaf area with atypical symptoms that included chlorosis or necrosis. These ratings were reported as phytotoxicity. Plots were not inoculated; infection was induced from natural field inocula. Final percent disease data were analyzed with JMP-SAS for analysis of variance and significant differences. Tukey's HSD was used to assess multiple comparisons of means (0.05).

All AX formulations were made using the production method described in Formulation H and I. The formulations were as follows:

### Formulations

4/0.8 mM KI/KSCN, 50 mM glucose, 10 mM phosphate, 10 U/ml GOx, 12 U/ml LP
4.0 mM KI, 50 mM glucose, 10 mM phosphate, 10 U/ml GOx, 12 U/ml LP

The weather was extremely favorable for the development of downy mildew and resulted in severe disease pressure. Both AX treatments reduced downy mildew disease severity when compared to the control and were not significantly different from each other. The AX treatment with both 4 mM KI, and 0.8 mM KSCN was slightly more effective than the treatment with 4 mM KI alone. No phytotoxicity from AX formulations were observed (FIG. 29). These data surprisingly show that KSCN was not required for disease suppression.

### Example 21: Disease Suppression of Early Blight of Tomato

Early tomato blight caused by the fungal pathogen, Alternaria solani, is an extremely destructive pathogen. A replicated small plot trial was conducted to evaluate selected fungicides for control of early blight on tomatoes. Tomato CV ‘Mt. Fresh” plants were transplanted in the field in a Howard gravelly loam soil on Jul. 11, 2023. The trial was a completely randomized block design with four replications of each treatment and a nontreated control. Commercial fungicides, Champ 2F and Regalia as well as AX treatments were applied five times at a rate of 25 gallons per acre (gpa) with a CO₂ hand-held pressurized (37 psi) sprayer 43, 52, 58, 65, and 71 DAP. The spray boom consisted of 2 hollow cone nozzles (=TeeJet TXVS18) set on the 18-inch center. Plants were rated for the percent leaf area with symptoms of early blight on 50, 58, 64, and 72 DAP. Plots were not inoculated; infection was induced from natural field inoculum. Final percent disease data were analyzed with JMP-SAS for analysis of variance and significant differences. Tukey's HSD was used to assess multiple comparison of means (0.05).

All AX formulations were made using the production method described in Formulation E, F and G. The formulations were as follows:

### Formulations

4/0.8 mM KI/KSCN, 50 mM glucose, 10 mM phosphate, 10 U/ml GOx, 12 U/ml LP
4/0.8 mM KI/KSCN, 50 mM glucose, 10 mM phosphate, 10 U/ml GOx
4.0 mM KI, 50 mM glucose, 10 mM phosphate, 10 U/ml GOx, 12 U/ml LP

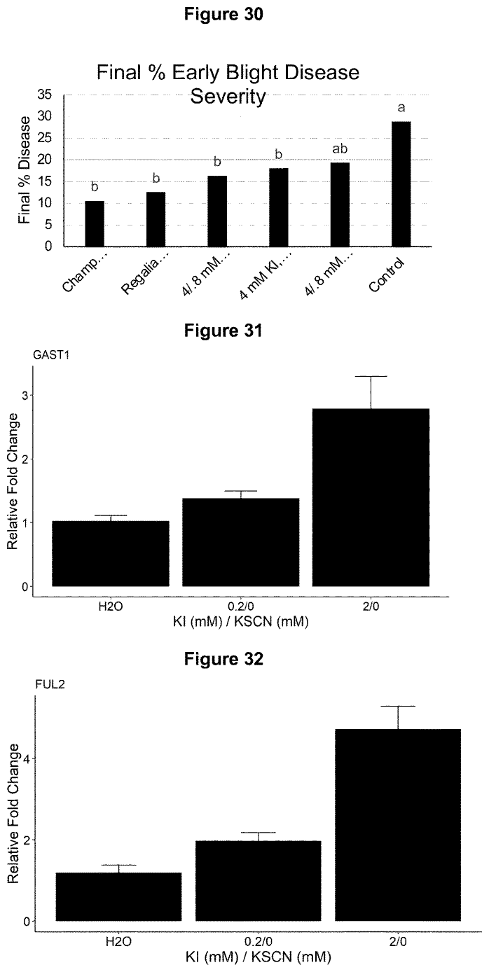

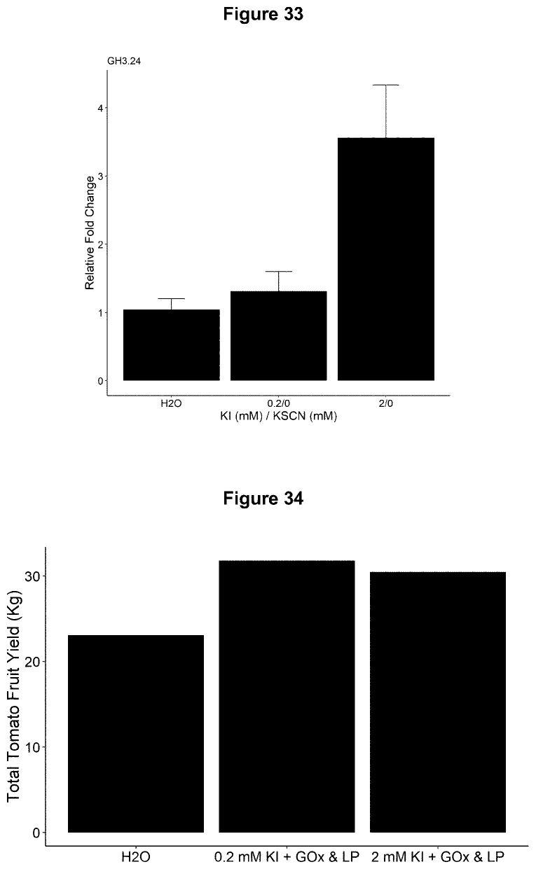

All three AX treatments reduced early blight disease severity when compared to the non-treated control and were not significantly different from each other. The AX treatment with both GOx and LP was slightly more effective than the treatment with GOx alone. No phytotoxicity from AX formulations were observed. (FIG. 30.)

### Example 22: Inducing Biostimulant Response Gene Activation in Tomato

Tomato plants (cultivar Mt. Fresh) were started in a greenhouse and moved to a field 39 days later. There were six replications per treatment. A total of ten applications were made with a hand held sprayer on 23, 30, 37, 44, 51, 58, 79, 86, 93, and 100 days after planting. In the first six applications, 1 ml was applied. In the final four applications, 2 ml were applied. One day post-application, samples were taken and analyzed for induced expression of genes involved in regulation of flowering Gibberellic Acid Stimulated Transcript 1 (GAST1), fruit development Fruitfull-Like 2 (FUL2), quality Gretchen Hagen 3.24 (GH3.24), flowering time, and fruit ripening in tomato when compared to water controls.

Two leaflets were sampled from the second-youngest leaves as a single biological replicate. Leaf tissue was homogenized via mortar and pestle in QIAGEN RLT buffer with B-mercaptoethanol at a 1:100 dilution. Total RNA was extracted from homogenized leaf tissue with the Monarch Total RNA Miniprep Kit (T2010S) from New England BioLabs following standard manufacturer protocols. Total RNA concentration was determined with a Nanodrop spectrophotometer.

Gene expression was determined through Reverse Transcription-quantitative Polymerase Chain Reaction (RT-qPCR) with the LUNA Universal One-Step RT-qPCR Kit (E3005L) from New England BioLabs following manufacturer protocols. RT-qPCR was run on a QuantStudio 7 Pro and the resulting data was analyzed via the 2-delta CT method in the Design and Analysis App (DA2) from ThermoFisher Scientific. Relative fold-change compared the expression of target genes to an internal tubulin control gene within each group and the relative change observed between the target and control gene between a given treatment group and the water control. Primers are shown in FIG. 19.

All AX formulations were made using the production method described in Formulation J. Formulations were as follows:

### Formulations

0.2 mM KI, 50 mM glucose, 10 mM phosphate, 2 U/ml GOx, 3 U/ml LP
2 mM KI, 50 mM glucose, 10 mM phosphate, 5 U/ml GOx, 6 U/ml LP

Both AX formulations upregulated GAST1, FUL2, and GH3.24. The 2 mM KI treatment, however, induced significantly higher gene expression than the 0.2 mM KI formulation (Figures, 31, 32 and 33).

### Example 23: AX Biostimulant Response Increased Tomato Yield

Tomato plants (cultivar Mt. Fresh) were started in a greenhouse and moved to the field 39 days later. A total of ten applications were made with a handheld sprayer on 23, 30, 37, 44, 51, 58, 79, 86, 93, and 100 days after planting. The first six applications consisted of 1 ml and the final four consisted of 2 ml. There were six replications per treatment. All AX formulations were made using the production method described in Formulation J. Formulations were as follows:

### Formulations

0.2 mM KI, 50 mM glucose, 10 mM phosphate, 2 U/ml GOx, 3 U/ml LP 2 mM KI, 50 mM glucose, 10 mM phosphate, 5 U/ml GOx, 6 U/ml LP

Tomato harvest began on 107 DAP and concluded 157 DAP. These data show the AX treatments with 2 mM KI and 0.2 mM KI had higher yields than the water control (FIG. 34).

All publications and patent documents disclosed or referred to herein are incorporated by reference in their entirety. The foregoing description has been presented only for purposes of illustration and description. This description is not intended to limit the invention to the precise form disclosed. It is intended that the scope of the invention be defined by the claims appended hereto.

1. A composition, comprising a hydrogen peroxide source and an iodide source, wherein said composition is optimized for inducing a Systemic Acquired Resistance (SAR) in a plant.

2. The composition of claim 1, wherein said hydrogen peroxide source is dissolved hydrogen peroxide.

3. The composition of claim 1, wherein said hydrogen peroxide source is glucose oxidase (GOx) and glucose.

4. The composition of claim 3, wherein said GOx is at a concentration of about 1, 2, 3, 4, 5, 6, 7, 8, 9, 10, 11, 12, 13, 14, 15, 16, 17, 18, 19, 20, 30, 40, or 50 U/ml.

5. The composition of claim 4, wherein said GOx is at a concentration of about 10 U/ml.

6. The composition of claim 3, wherein said GOx is at a concentration of 20 U/ml.

7. The composition of claim 3, wherein said GOx is at a concentration of 40 U/ml.

8. The composition of claim 3, wherein said glucose is at a concentration of about 2, 4, 6, 8, 10, 20, 30, 40, 50, 60, 70, 80, 90, 100, 200, 300, 400, or 500 mM.

9. The composition of claim 3, wherein said glucose is at a concentration of 50 mM.

10. The composition of claim 1, wherein said iodide source is an iodide salt.

11. The composition of claim 10, wherein said iodide salt is KI, NaI, or NH4I.

12. The composition of claim 11, wherein said iodide salt is KI.

13. The composition of claim 10, wherein said iodide salt has a concentration of about 0.05, 0.1, 0.2, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 20.0, 30.0, 35.0, 40.0, 50.0, 60.0, 70.0, 80.0, 90.0, or 100.0 mM.

14. The composition of claim 13, wherein said iodide salt has a concentration of about 2 mM.

15. The composition of claim 13, wherein said iodide salt is KI.

16. The composition of claim 14, wherein said KI is at a concentration of about 2 mM.

17. The composition of claim 10, wherein said iodide salt has a concentration of about 2 mM.

18. The composition of claim 10, wherein said iodide salt has a concentration of about 4 mM.

19. The composition of claim 10, wherein said iodide salt has a concentration of about 18 mM.

20. The composition of claim 10, wherein said iodide salt has a concentration of about 35 mM, 40 mM, or 70 mM.

21. The composition of claim 1, wherein said composition does not comprise lactoperoxidase.

22. The composition of claim 1, wherein said composition does not comprise a thiocyanate source.

23. The composition of claim 1, further comprising a thiocyanate source.

24. The composition of claim 23, wherein said thiocyanate source is a thiocyanate salt or an organic thiocyanate.

25. The composition of claim 24, wherein said thiocyanate salt is KSCN, NaSCN, or NH4SCN.

26. The composition of claim 24, wherein said thiocyanate salt is KSCN.

27. The composition of claim 24, wherein said thiocyanate salt has a concentration of about 0.5, 0.6, 0.7, 0.8, 0.9, 1.0, 1.5, 2.0, 2.5, 3.0, or 3.5 mM.

28. The composition of claim 24, wherein said thiocyanate salt has a concentration of about 4 mM.

29. The composition of claim 24, wherein said thiocyanate salt has a concentration of about 8 mM or 16 mM.

30. The composition of claim 15, wherein said hydrogen peroxide source is GOx at a concentration of 20 U/ml and glucose at a concentration of 300 mM, and said KI is at a concentration of 70 mM.

31. The composition of claim 15, wherein said hydrogen peroxide source is GOx at a concentration of 40 U/ml and glucose at a concentration of 300 mM, and said KI is at a concentration of 70 mM.

32. The composition of claim 15, wherein said hydrogen peroxide source is GOx at a concentration of 20 U/ml and glucose at a concentration of 300 mM, and said KI is at a concentration of 18 mM.

33. The composition of claim 15, wherein said hydrogen peroxide source is GOx at a concentration of 20 U/ml and glucose at a concentration of 300 mM, and said KI is at a concentration of 35 mM.

34. The composition of claim 15, wherein said hydrogen peroxide source is GOx at a concentration of 40 U/ml and glucose at a concentration of 300 mM, and said KI is at a concentration of 35 mM.

35. The composition of claim 15, wherein said hydrogen peroxide source is GOx at a concentration of 10 U/ml and glucose at a concentration of 50 mM, and said KI is at a concentration of 2 mM.

36. The composition of claim 15, wherein said hydrogen peroxide source is GOx at a concentration of 10 U/ml and glucose at a concentration of 50 mM, and said KI is at a concentration of 4 mM.

37. The composition of claim 1, wherein said composition acts as a biostimulant.

38. The composition of claim 1, wherein said composition directly kills microbes.

39. The composition of claim 1, further comprising lactoperoxidase (LP).

40. The composition of claim 39, wherein said LP is at a concentration of about 1, 3, 5, 10, 11, 12, 13, 14, 15, 16, 17, 18, 19, 20, 21, 22, 23, 24, 25, 26, 27, 28, 29, or 30 U/ml.

41. The composition of claim 40, wherein said LP is at a concentration of about 35, 40, 45, or 50 U/ml.

42. The composition of claim 1, wherein said composition is dormant and become active upon exposure to hydration, oxygen, or mixing.

43. A method of inducing Systemic Acquired Resistance (SAR) in a plant, comprising the step of administering the composition of claim 1 at a rate that is optimized for SAR induction.

44. The method of claim 43, wherein said composition is administered by coating, spraying, sprinkling, atomizing, overhead spraying, watering, immersing, overhead irrigation, or drip irrigation.

45. The method of claim 44, wherein said composition is administered by spraying.

46. The method of claim 43, wherein said plant is selected from the group consisting of vegetable, fruit, flower, and field crop.

47. The method of claim 46, wherein said vegetable plant is selected from the group consisting of tomato, pea, onion, garlic, parsley, oregano, basil, cilantro, carrot, cabbage, cucumber, radish, pepper, broccoli, cauliflower, spinach, kale, chard, artichoke, and lettuce.

48. The method of claim 46, wherein said vegetable plant is tomato.

49. The method of claim 46, wherein said vegetable plant is cabbage.

50. The method of claim 46, wherein said vegetable plant is cucumber.

51. The method of claim 46, wherein said fruit plant is selected from the group consisting of citrus, tomato, orange, lemon, lime, avocado, clementine, apple, persimmon, pear, peach, nectarine, berry, strawberry, raspberry, grape, blueberry, blackberry, cherry, apricot, gourds, squash, zucchini, eggplant, pumpkin, coconut, guava, mango, papaya, melon, honeydew, cantaloupe, watermelon, banana, plantain, pineapple, quince, sorbus, loquata, plum, currant, pomegranate, fig, olive, fruit pit, a nut, peanut, almond, cashew, hazelnut, brazil nut, pistachio, and macadamia.

52. The method of claim 51, wherein said fruit plant is pumpkin.

53. The method of claim 46, wherein said flower plant is selected from the group consisting of annual, perennial, bulb, flowering woody stem, carnation, rose, tulip, poppy, snapdragon, lily, mum, iris, alstroemeria, pom, fuji, and bird of paradise.

54. The method of claim 46, wherein said field crop is selected from the group consisting of corn, wheat, soybean, canola, sorghum, potato, sweet potato, yam, lentils, beans, snap beans, cassava, coffee, hay, buckwheat, oat, barley, rape, switchgrass, elephant grass, beet, sugarcane, and rice.

55. The method of claim 46, wherein said field crop is corn.

56. The method of claim 46, wherein said field crop is wheat.

57. The method of claim 46, wherein said field crop is potato.

58. The method of claim 46, wherein said field crop is snap beans.

59. The method of claim 43, wherein said compositions are administered to plant leaves.

60. The method of claim 43, wherein said compositions are administered to seeds.

61. A method for inducing iodine fortification in a plant, comprising the step of administering the composition of claim 1 at a rate that is optimized for increasing iodine uptake or retention and induction.

62. The method of claim 60, wherein said composition is administered by coating, spraying, sprinkling, atomizing, overhead spraying, watering, immersing, overhead irrigation, or drip irrigation.

63. The method of claim 62, wherein said composition is administered by spraying.

64. The method of claim 61, wherein said plant is selected from the group consisting of vegetable, fruit, flower, and field crop.

65. The method of claim 64, wherein said vegetable plant is selected from the group consisting of tomato, pea, onion, garlic, parsley, oregano, basil, cilantro, carrot, cabbage, cucumber, radish, pepper, broccoli, cauliflower, spinach, kale, chard, artichoke, and lettuce.

66. The method of claim 64, wherein said vegetable plant is tomato.

67. The method of claim 64, wherein said vegetable plant is cabbage.

68. The method of claim 64, wherein said vegetable plant is cucumber.

69. The method of claim 64 wherein said fruit plant is selected from the group consisting of citrus, tomato, orange, lemon, lime, avocado, clementine, apple, persimmon, pear, peach, nectarine, berry, strawberry, raspberry, grape, blueberry, blackberry, cherry, apricot, gourds, squash, zucchini, eggplant, pumpkin, coconut, guava, mango, papaya, melon, honeydew, cantaloupe, watermelon, banana, plantain, pineapple, quince, sorbus, loquata, plum, currant, pomegranate, fig, olive, fruit pit, a nut, peanut, almond, cashew, hazelnut, brazil nut, pistachio, and macadamia.

70. The method of claim 64, wherein said fruit plant is pumpkin.

71. The method of claim 66, wherein said flower plant is selected from the group consisting of annual, perennial, bulb, flowering woody stem, carnation, rose, tulip, poppy, snapdragon, lily, mum, iris, alstroemeria, pom, fuji, and bird of paradise.

72. The method of claim 66, wherein said field crop is selected from the group consisting of corn, wheat, soybean, canola, sorghum, potato, sweet potato, yam, lentils, beans, snap beans, cassava, coffee, hay, buckwheat, oat, barley, rape, switchgrass, elephant grass, beet, sugarcane, and rice.

73. The method of claim 64, wherein said field crop is corn.

74. The method of claim 64, wherein said field crop is wheat.

75. The method of claim 64, wherein said field crop is potato.

76. The method of claim 64 wherein said field crop is snap beans.

77. The method of claim 61, wherein said compositions are administered to plant leaves.

78. The method of claim 61, wherein said compositions are administered to seeds.

79. A method of manufacturing an enzyme-free composition, comprising:
a) immobilizing an H2O2-producing enzyme and a free radical producing (FRP) enzyme in a reaction container,
b) exposing said immobilized H2O2-producing and free radical producing (FRP) enzymes to a reaction solution comprising an iodide source, a thiocyanate source, and a substrate for said H2O2-producing enzyme; and
c) collecting a product solution, wherein said solution comprises reactive oxidative species.

80. The method of claim 76, wherein said H2O2-producing enzyme is glucose oxidase (GOx) and said substrate is glucose.

81. The method of claim 76, wherein said FRP enzyme is lactoperoxidase.

82. The method of claim 79, wherein said iodide source is KI.

83. The method of claim 79, wherein said thiocyanate source is KSCN.

84. The method of claim 80, wherein said glucose is at a concentration of 300 mM in said solution.

85. The method of claim 82, wherein said KI is at a concentration of 35 mM in said solution.

86. The method of claim 83, wherein said KSCN is at a concentration of 8 mM in said solution.

87. The method of claim 79, wherein said product solution comprises one or more reactive oxidative species selected from the group consisting of I2, I3-, I2SCN-, and I(SCN)2-.

88. The method of claim 79, wherein said H2O2-producing enzyme and FRP enzyme are immobilized on hematite.

89. The method of claim 79, wherein said reaction container is a packed bed reactor.

90. The method of claim 89, wherein said reaction solution is pumped with a high-pressure pump through said packed bed reactor.
